(19) United States
(12) Patent Application Publication
(10) Pub. No.: US 2017/0030898 A1
(43) Pub. Date: Feb. 2, 2017

(54) Characterization and Directed Evolution of a Methyl Binding Domain Protein for High-Sensitivity DNA Methylation Analysis

(71) Applicant: MASSACHUSETTS INSTITUTE OF TECHNOLOGY, CAMBRIGE, MA (US)

(72) Inventors: BRANDON W. HEIMER, CHARLESTOWN, MA (US); BROOKE E. TAM, CAMBRIDGE, MA (US); HADLEY D. SIKES, ARLINGTON, MA (US)

(21) Appl. No.: 15/190,980

(22) Filed: Jun. 23, 2016

Related U.S. Application Data
(60) Provisional application No. 62/183,479, filed on Jun. 23, 2015.

(51) Int. Cl. G01N 33/53; C07K 14/47; C12Q 1/68

(52) U.S. Cl. CPC G01N 33/5308; C12Q 1/6876; G01N 33/5306; C07K 14/47; C12Q 2600/154; C12Q 2600/158; C12Q 2600/156

(57) ABSTRACT

This present invention provides high affinity variants of human methyl binding domain 2 (hMBD2), and nucleic acids encoding the variants, capable of recognizing and/or binding to methylated DNA. In particular, the hMBD2 variants of the invention recognize and/or bind a DNA sequence with single methylated CpG site with high affinity. The invention provides materials and methods for using the nucleic acid and/or amino acid sequence variants hMBD2 of the invention to detect methylated DNA. The hMBD2 variants of the invention are particularly useful for recognizing and/or binding a DNA sequence with single methylated CpG site with high affinity.

### RELATED APPLICATION

This application claims the benefit of U.S. Provisional Application No. 62/183,479, filed on Jun. 23, 2015. The entire teachings of the above application are incorporated herein by reference.

### GOVERNMENT FUNDING

This invention was made with Government support under Grant No. P30 ES002109 awarded by the National Institutes of Health. The Government has certain rights in the invention.

### BACKGROUND

The structure of chromatin plays a significant role in gene expression and development for eukaryotic organisms (Hashimshony et al., 2003). Methylation at the 5 position of the cytosine base, when followed by guanine (CpG) in the promoter region of a protein-coding gene, is an epigenetic modification that has been shown to be involved in DNA condensation and transcriptional inactivation (Wolffe and Matzke, 1999). Aberrant DNA methylation patterns have been implicated in the development of human diseases such as cancer (Feinberg, 2007). Medical research has connected promoter methylation levels for certain genes to therapeutic response in patients. For example, glioma patients with a methylated promoter for the O⁶-methylguanine-DNA methyltransferase (MGMT) gene exhibit particular sensitivity to alkylating agent chemotherapeutics (Hegi et al., 2005), and breast cancer patients with methylation-dependent silencing of the breast cancer 1, early onset (BRCA1) gene have been shown to have tumors sensitive to cisplatin (Silver et al., 2010). Additionally, physicians can test for epigenetic silencing of the DNA mismatch repair gene MutL homolog 1 (MLHJ) for its prognostic value for patients being treated with colon cancer (Herman et al., 1998, Heyn and Esteller, 2012). Hypermethylation at glutathione S-transferase pi 1 (GSTP1) has also shown promise as a biomarker for diagnosing prostate cancer (Van Neste et al., 2012).

Because promoter methylation has been shown to have predictive, prognostic and diagnostic value, there has been great interest in developing methods for DNA methylation detection with increased sensitivity, specificity, and resolution to increase clinical value (Heyn and Esteller, 2012) and also for discovery purposes to generate reference methylome data (Roadmap Epigenomics et al., 2015).

State of the art methods for DNA methylation detection (whole-genome bisulfite sequencing, reduced representation bisulfite sequencing, CpG specific arrays, and methylation-specific PCR) generally rely on sodium bisulfite conversion of unmethylated cytosine bases to uracil (Heyn and Esteller, 2012). Chemical conversion, however, can degrade more than 90% of the sample DNA (Grunau et al., 2001), and protocols must be assiduously optimized to minimize incomplete deamination of unmethylated cytosine bases and inappropriate conversion of methylated ones to thymine (Genereux et al., 2008). Such errors lead to inaccurate results. Alternatively, immunoprecipitation (IP) based methods such as MeDIP-seq and MBD-seq have been developed. These methods tend to require larger sample inputs (Laird, 2010) and are not capable of providing single methyl CpG site resolution without bisulfite conversion (Pomraning et al., 2009).

To avoid bisulfite conversion while still providing improved resolution, there have been several methods developed recently that use the very methyl binding domain (MBD) proteins involved in forming repressive complexes in vivo to transduce DNA methylation into a signal that can be measured directly (Cipriany et al., 2012, Cipriany et al., 2010, Heimer et al., 2014, Luo et al., 2009, Yu et al., 2010) instead of simply providing sample enrichment as is the case with MBD-seq. These MBD proteins specifically recognize symmetrically methylated CpG dinucleotides in double stranded DNA (Fraga et al., 2003, Hendrich and Bird, 1998, Jorgensen et al., 2006), and therefore, have the potential to enable high resolution DNA methylation detection when paired with sequence specific probe DNA without requiring chemical conversion or sequencing of DNA.

Current MBD-based methods require relatively large amounts of DNA (Heimer, Shatova, Lee, Kaastrup and Sikes, 2014, Luo, Zheng, Wang, Wu, Bai and Lu, 2009, Yu, Blair, Gillespie, Jensen, Myszka, Badran, Ghosh and Chagovetz, 2010) or are not sequence specific (Cipriany, Murphy, Hagarman, Cerf, Latulippe, Levy, Benitez, Tan, Topolancik, Soloway and Craighead, 2012, Cipriany, Zhao, Murphy, Levy, Tan, Craighead and Soloway, 2010). Clinical applications require that both these problems be addressed (Heyn and Esteller, 2012).

Thus there is a need for a very high affinity MBD protein suitable for interfacial use and capable of recognizing a single methylated CpG site. Such a MBD protein will thermodynamically provide a higher fractional coverage of these sites in DNA (Kaastrup et al., 2013), which is particularly important when the total number of sites may be low. Such a reagent would support ongoing research to make methylation analysis on a single DNA molecule (Cipriany, Murphy, Hagarman, Cerf, Latulippe, Levy, Benitez, Tan, Topolancik, Soloway and Craighead, 2012, Cipriany, Zhao, Murphy, Levy, Tan, Craighead and Soloway, 2010, Shapiro et al., 2013, Wang and Bodovitz, 2010) sequence specific.

### SUMMARY OF THE INVENTION

This present invention provides high affinity variants of human methyl binding domain 2 (hMBD2), and nucleic acids encoding the variants, capable of recognizing and/or binding to methylated DNA. In particular, the hMBD2 variants of the invention recognize and/or bind a DNA sequence with single methylated CpG site with high affinity. The invention provides materials and methods for using the nucleic acid and/or amino acid sequence variants hMBD2 of the invention to detect methylated DNA. The hMBD2 variants of the invention are particularly useful for recognizing and/or binding a DNA sequence with single methylated CpG site with high affinity.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1—Synthetic DNA oligonucleotides derived from the MGMT gene. All oligos have the same sequence containing three CpG dinucleotides. The schematic shows the location and number of methylated CpGs for each test oligo. A 5′ biotin was appended to one strand to facilitate detection using a streptavidin conjugated fluorophore. (SEQ ID NO: 2 and SEQ ID NO: 3, respectively)

FIG. 2—Sequencing results of the pCTCON-2/MBD constructs. The DNA encoding the HA-MBD-c-Myc sequence and corresponding amino acid translation are shown for hMBD2 displayed on EBY 100 yeast cells.

FIG. 3a and FIG. 3b—Amino acid sequences of unique MBD variants isolated from the first (a) (SEQ ID NOs: 6-11, respectively) and second (b) (SEQ ID NO: 6 and SEQ ID NOs: 12-15, respectively) rounds of epPCR and screening.

FIG. 4a and FIG. 4b—A reduced resolution, five-point equilibrium binding experiment was used to quickly screen the relative methylated DNA binding affinities of unique MBD variants relative to wild-type hMBD2 after the first (a) and second (b) rounds of epPCR. The variants from each round with the highest apparent binding affinity were selected for complete equilibrium binding titrations to omo DNA in triplicate to quantitatively determine K_{d}.

FIG. 5a through FIG. 5e—Detection and quantification of methylated DNA binding to yeast displayed MBD proteins. (a) Yeast displaying MBD proteins were incubated with biotinylated, methylated DNA and a primary anti-c-Myc antibody followed by labeling with streptavidin, ALEXA FLUOR® 647 and an ALEXA FLUOR® 488 secondary antibody, respectively. (b) Flow cytometry dot plot showing 50 nM omo DNA and (c) 50 nM ooo DNA binding to wild-type hMBD2. (d) Equilibrium binding titration curves for determining the affinity of wild-type hMBD2 binding to DNA with various DNA methylation patterns. The mean fluorescence of the displaying yeast population is normalized and plotted versus DNA concentration. Fitting the data yields the equilibrium dissociation constant (K_{d}) for each oligo. Each reported value (Table I) is the average of three such biological replicates (only one shown). (e) Titration curves for wild-type MBD2, variant 1/4, and variant 2/5 binding to omo DNA. Leftward shift of the binding curve indicates higher affinity binding.

FIG. 6—Sequence comparison of MBD2 proteins. The MBD variants 1/4 and 2/5 having two and five mutations, respectively, are shown below the wild-type hMBD2 sequence. The sequence of chicken MBD2 is included for reference, and its secondary structure, determined from previous NMR analysis, is depicted above the sequence alignment.

FIG. 7a through FIG. 7d.—Structural analysis of amino acid substitutions in MBD variant 2/5. (a) The addition of the para substituted hydroxyl group of tyrosine relative to the wild-type phenylalanine forms a new hydrogen bond to the DNA phosphodiester backbone. (b) Mutating lysine 161 to arginine introduces a guanidinium group capable of forming an additional hydrogen bond to the main chain carbonyl of aspartic acid 151 in addition to that the wild-type lysine makes to the main chain carbonyl of glycine 211. (c). The side chains of isoleucines 165 and 175 form a hydrophobic interaction at the end of 32 and beginning of 33. (d) Isoleucine 187 and leucine 193 share a hydrophobic interaction between 04 and al in the vicinity of three residues lysine 186 (backbone), arginine 188 (bases), and serine 189 (backbone) known to interact with the bound DNA strand.

FIG. 8a through FIG. 8c-N×MBD2-Var2/5-GFP proteins bind to surface-immobilized DNA. (a) Fluorescent scan of 60 nM 1×MBD2-Var2/5-GFP binding to omm, omo, and ooo DNA on a biochip using an anti-HA/ALEXA FLUOR®647 antibody pair for detection. (b) Titration curves were fitted to plots of the mean fluorescence from DNA spots versus the concentration of 1×MBD2-Var2/5-GFP (b) or 3×MBD2-Var2/5-GFP (c) applied to the array to determine the apparent dissociation constant K_{d,app} of each reagent for interfacial binding.

FIG. 9—Modeling result for the fraction of CpG dinucleotides with bound MBD over various concentrations of MBDs with equilibrium dissociation constants ranging from 10⁻¹⁰ to 10⁻⁶ M (0.1-1000 nM). Higher binding affinities and higher MBD concentrations favor higher CpG fractional coverages.

FIG. 10a through FIG. 10c—Characterization of binding affinity of wild-type MBD2 for hemi-methylated DNA. a) The DNA sequence of the probe/target pairs and methylation states used for protein assessment are shown with the methylated cytosine bases bolded and underlined. The sequence is from the MGMT gene and contains three CpG dinucleotides, one of which is methylated in the hemi-methylated and symmetrically methylated states used in this paper. b) An equilibrium binding titration of wild-type MBD2. The fraction of MBD bound to DNA was determined based on the normalized mean fluorescence. Wild-type MBD2 binds symmetrically methylated DNA with high specificity and shows no detectable binding to hemi-methylated DNA at the concentrations of interest. c) Reported dissociation constants for MBD1 and MeCP2 also show affinity differences of one or more orders of magnitude between symmetrically methylated and hemi-methylated DNA.

FIG. 11—Schematic of the process for selecting hMBD2 variants of the invention having affinity for hemi-methylated DNA using an equilibrium binding assay. MBD variants were expressed on the surface of S. cerevisiae. Cells expressing variants with improved affinity for hemi-methylated DNA were selected first with magnetic beads coated in hemi-methylated DNA and then with fluorescently labeled hemi-methylated DNA and fluorescence activated cell sorting.

FIG. 12a and FIG. 12b—Characterization of variant h4 by equilibrium binding titrations. a) An equilibrium binding titration shows the binding affinity for the engineered variant H4 in comparison with the wild-type hMBD2 protein. b) The engineered hMBD2 protein H4 is characterized by equilibrium binding titrations with hemi-methylated, symmetrically methylated, and unmethylated DNA.

FIG. 13a and FIG. 13b—hMBD2 variants of the invention can distinguish between hemi-methylated and unmethylated DNA. a) An image of the DNA array after labeling of MBD2 Variant H4 shows that the hemi-methylated and unmethylated spots are easily distinguishable. b) Quantitative analysis shows a 7.8-fold higher signal from binding to hemi-methylated DNA as compared to unmethylated DNA in the arrays.

### DETAILED DESCRIPTION OF THE INVENTION

Provided herein are isolated human methyl bind domain 2 (hMBD2) nucleic acid and amino acid sequence variants. The hMBD2 variants of the invention bind methylated DNA. In particular, the hMBD2 variants of the invention recognize and/or bind DNA comprising a single methylated CpG site, with high affinity. The hMBD2 nucleic acid sequence variants are relative to the reference wild-type hMBD2 sequence (GAAAGCGGCAAACGCATGGATTGCCCGGCGCTGCCGCCGGGTTGGAAAAAAGAAG AAGTGATTCGTAAAAGCGGCCTGAGCGCGGGCAAAAGCGATGTGTATTATTTTAGC CCGAGCGGCAAAAAATTTCGTAGCAAACCGCAGCTGGCGCGTTATCTGGGCAACAC CGTGGATCTGAGCAGCTTTGATTTTCGTACCGGCAAAATG/SEQ ID NO: 16). The hMBD2 amino acid sequence variants are relative to the reference wild-type hMBD2 amino acid sequence (ESGKRMDCPALPPGWKKEEVIRKSGLSAGKSDVYYFSPSGKKFRSKPQLARYLGNTVD LSSFDFRTGKM/SEQ ID NO: 6).

Units, prefixes, and symbols can be denoted in the SI accepted form. Numeric ranges are inclusive of the numbers defining the range. Unless otherwise indicated, nucleic acids are written left to right in 5′ to 3′ orientation, respectively. The headings provided herein are not limitations of the various aspects or embodiments of the invention which can be had by reference to the specification as a whole. Accordingly, the terms defined immediately below are more fully defined by reference to the specification as a whole.

“About” as used herein means that a number referred to as “about” comprises the recited number plus or minus 1-10% of that recited number. For example, “about” 50 nucleotides can mean 45-55 nucleotides or as few as 49-51 nucleotides depending on the situation. Whenever it appears herein, a numerical range, such as “45-55”, refers to each integer in the given range; e.g., “45-55 nucleotides” means that the nucleic acid can contain 45 nucleotides, 46 nucleotides, etc., up to and including 55 nucleotides.

The terms “oligonucleotide”, “polynucleotide” and “nucleic acid (molecule)” are used interchangeably to refer to polymeric forms of nucleotides of any length. The polynucleotides may contain deoxyribonucleotides, ribonucleotides and/or their analogs. Nucleotides may be modified or unmodified and have any three-dimensional structure, and may perform any function, known or unknown. The term “polynucleotide” includes single-, double-stranded and triple helical molecules. Oligonucleotides are also known as oligomers or oligos and may be isolated from genes, or chemically synthesized by methods known in the art.

Polynucleotide sequences can be considered to be substantially identical if two molecules hybridize to each other under stringent conditions. However, polynucleotides which do not hybridize to each other under stringent conditions are still substantially identical if the polypeptides which they encode are substantially identical. This can occur when a copy of a polynucleotide is created using the maximum codon degeneracy permitted by the genetic code.

As used herein, “isolated nucleic acid” refers to a nucleic acid that is separated from other nucleic acid molecules that are present in a mammalian genome, including nucleic acids that normally flank one or both sides of the nucleic acid in a mammalian genome (e.g., nucleic acids that encode non-RBM20 proteins). The term “isolated” as used herein with respect to nucleic acids also includes any non-naturally-occurring nucleic acid sequence since such non-naturally-occurring sequences are not found in nature and do not have immediately contiguous sequences in a naturally-occurring genome. An isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid.

A “primer” refers to an oligonucleotide containing at least 6 nucleotides, usually single-stranded, that provides a 3′-hydroxyl end for the initiation of enzyme-mediated nucleic acid synthesis. A “polynucleotide probe” is a polynucleotide that specifically hybridizes to a complementary polynucleotide sequence.

As used herein, the terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation.

As used herein, the term “conservatively modified variants” applies to both amino acid and nucleic acid sequences. With respect to particular nucleic acid sequences, conservatively modified variants refers to those nucleic acids which encode identical or conservatively modified variants of the amino acid sequences. Because of the degeneracy of the genetic code, a large number of functionally identical nucleic acids encode any given protein. For example, the codons GCA, GCC, GCG and GCU all encode the amino acid alanine. Thereupon, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein which encodes a polypeptide also describes every possible “silent variation” of the nucleic acid. It is known by persons skilled in the art that each codon in a nucleic acid (except AUG, which is the only codon for the amino acid, methionine; and UGG, which is the only codon for the amino acid tryptophan) can be modified to yield a functionally identical molecule. Therefore, each silent variation of a nucleic acid which encodes a polypeptide of the present invention is implicit in each described polypeptide sequence. In some embodiments, a nucleotide sequence variant encodes a polypeptide having an altered amino acid sequence.

With respect to amino acid sequences, persons skilled in the art will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

“Transcription” as used herein, refers to the enzymatic synthesis of an RNA copy of one strand of DNA (i.e., template) catalyzed by an RNA polymerase (e.g. a DNA-dependent RNA polymerase).

A “target DNA sequence” is a DNA sequence of interest for which detection, characterization or quantification is desired. The actual nucleotide sequence of the target sequence may be known or not known. Target DNAs are typically DNAs for which the CpG methylation status is interrogated. A “target DNA fragment” is a segment of DNA containing the target DNA sequence. Target DNA fragments can be produced by any method including e.g., shearing or sonication, but most typically are generated by digestion with one or more restriction endonucleases.

The methylated target DNA fragment is typically generated from a sample containing genomic DNA by restriction enzyme digestion. Methods for preparing and digesting genomic DNA with restriction enzymes are well known in the art. Samples suitable for analysis according to the methods of the invention include but are not limited to biological, clinical and biopsy specimens, such as blood, sputum, saliva, urine, semen, stool, bodily discharges, exudates, or aspirates and tissue samples, such as biopsy samples.

The terms “complementary” or “complementarity” are used in reference to a first polynucleotide (which may be an oligonucleotide) which is in “antiparallel association” with a second polynucleotide (which also may be an oligonucleotide). As used herein, the term “antiparallel association” refers to the alignment of two polynucleotides such that individual nucleotides or bases of the two associated polynucleotides are paired substantially in accordance with Watson-Crick base-pairing rules. Complementarity may be “partial,” in which only some of the polynucleotides' bases are matched according to the base pairing rules. Or, there may be “complete” or “total” complementarity between the polynucleotides. Those skilled in the art of nucleic acid technology can determine duplex stability empirically by considering a number of variables, including, for example, the length of the first polynucleotide, which may be an oligonucleotide, the base composition and sequence of the first polynucleotide, and the ionic strength and incidence of mismatched base pairs.

As used herein, the term “hybridization” is used in reference to the base-pairing of complementary nucleic acids, including polynucleotides and oligonucleotides containing 6 or more nucleotides. Hybridization and the strength of hybridization (i.e., the strength of the association between the nucleic acids) is impacted by such factors as the degree of complementary between the nucleic acids, the stringency of the reaction conditions involved, the melting temperature (Tₘ) of the formed hybrid, and the G:C ratio within the duplex nucleic acid. Generally, “hybridization” methods involve annealing a complementary polynucleotide to a target nucleic acid (i.e., the sequence to be detected either by direct or indirect means). The ability of two polynucleotides and/or oligonucleotides containing complementary sequences to locate each other and anneal to one another through base pairing interactions is a well-recognized phenomenon.

As used herein, “MBP” means methyl binding protein. There are various methyl binding proteins that may be used in accordance with various embodiments described herein, and include but are not limited to, MBD1, MBD2, MBD4, MeCP272 and the Kaison protein family.

As used herein, “MBD” means methyl-CpG-binding domain.

As used herein, the term “promoter” refers to a region of DNA upstream from the start of transcription and involved in recognition and binding of RNA polymerase and other proteins to initiate transcription. A promoter can optionally include distal enhancers or repressor elements which can be located several thousand base pairs from the start site of transcription.

As used herein, the term “constitutive promoter” refers to a promoter which is active under most environmental conditions.

As used herein, the term “inducible promoter” refers to a promoter which is under environmental control. Examples of environmental conditions that may affect transcription by inducible promoters include anaerobic conditions or the presence of light.

As used herein, the term “operably linked” includes reference to a functional linkage between a promoter and a nucleic acid sequence, wherein the promoter sequence initiates and/or mediates transcription of the nucleic acid sequence. Generally, operably linked means that the polynucleotide sequences being linked are contiguous and, where necessary to join two protein coding regions, contiguous and in the same reading frame.

As used herein, the term “recombinant” includes reference to a cell, or nucleic acid, or vector, that has been modified by the introduction of a heterologous nucleic acid or the alteration of a native nucleic acid to a form not native to that cell, or that the cell is derived from a cell so modified. For example, recombinant cells express genes that are not found within the native (non-recombinant) form of the cell or express native genes that are otherwise abnormally expressed, under expressed or not expressed at all.

As used herein, the term “recombinant expression cassette” is a nucleic acid construct, generated recombinantly or synthetically, with a series of specified nucleic acid elements which permit transcription of a particular nucleic acid in a target cell. The expression vector can be part of a plasmid, virus, or nucleic acid fragment. Typically, the recombinant expression cassette portion of the expression vector includes a nucleic acid to be transcribed, and a promoter.

As used herein, the term, “specifically binds” includes reference to the preferential association of a ligand, in whole or part, with a particular target molecule (i.e., “binding partner” or “binding moiety” relative to compositions lacking that target molecule). It is, of course, recognized that a certain degree of non-specific interaction may occur between a ligand and a non-target molecule. Nevertheless, specific binding, may be distinguished as mediated through specific recognition of the target molecule. Typically, specific binding results in a much stronger association between the ligand and the target molecule than between the ligand and non-target molecule.

By “fusion protein”, “fusion polypeptide” or “fusion peptide” it is meant a protein composed of a plurality of protein components that while typically unjoined in their native state, are joined by their respective amino and carboxyl termini through a peptide linkage to form a single continuous polypeptide. “Protein” in this context includes proteins, polypeptides and peptides. Plurality in this context means at least two. It will be appreciated that the protein components can be joined directly or joined through a peptide linker/spacer as known to one skilled in the art. In addition, as outlined below, additional components such as fusion partners including targeting sequences, etc. may be used.

By “reporter protein” or “reporter tag” it is meant a protein that by its presence in or on a cell or when secreted in the media allow the cell to be distinguished from a cell that does not contain the reporter protein. Reporter genes fall into several classes, as outlined above, including, but not limited to, detection genes, indirectly detectable genes, and survival genes.

In a preferred embodiment, the reporter protein is a detectable protein. A “detectable protein” or “detection protein” (encoded by a detectable or detection gene) is a protein that can be used as a direct label; that is, the protein is detectable (and preferably, a cell comprising the detectable protein is detectable) without further manipulations or constructs. As outlined herein, preferred embodiments of screening utilize cell sorting (for example via FACS) to detect reporter (and thus peptide library) expression. Thus, in this embodiment, the protein product of the reporter gene itself can serve to distinguish cells that are expressing the detectable gene. In this embodiment, suitable detectable genes include those encoding autofluorescent proteins.

As is known in the art, there are a variety of autofluorescent proteins known; these generally are based on the green fluorescent protein (GFP) from Aequorea and variants thereof; including, but not limited to, GFP, (Chalfie et al., “Green Fluorescent Protein as a Marker for Gene Expression,” Science 263(5148):802-805 (1994)); enhanced GFP (EGFP; Clontech—Genbank Accession Number U55762)), blue fluorescent protein (BFP; Quantum Biotechnologies, Inc., 1801 de Maisonneuve Blvd. West, 8th Floor, Montreal (Quebec) Canada H3H 1J9; Stauber, R. H., Biotechniques 24(3):462-471 (1998); Heim, R. and Tsien, R. Y. Curr. Biol. 6:178-182 (1996)), enhanced yellow fluorescent protein (EYFP; Clontech Laboratories, Inc., Palo Alto, Calif.) and red fluorescent protein. In addition, there are recent reports of autofluorescent proteins from Renilla and Ptilosarcus species. See WO 92/15673; WO 95/07463; WO 98/14605; WO 98/26277; WO 99/49019; U.S. Pat. Nos. 5,292,658; 5,418,155; 5,683,888; 5,741,668; 5,777,079; 5,804,387; 5,874,304; 5,876,995; and 5,925,558; all of which are expressly incorporated herein by reference.

As used herein, the term “sample” refers to any biological sample obtained from a subject or an individual, cell line, tissue culture, or other source containing polynucleotides or polypeptides or portions thereof. As indicated, biological samples include body fluids (such as blood, sera, plasma, urine, synovial fluid and spinal fluid) and tissue sources found to express the polynucleotides of the present invention. Methods for obtaining tissue biopsies and body fluids from mammals are well known in the art. A biological sample which includes genomic DNA, mRNA or proteins is preferred as a source.

The present invention provides a variant human methyl binding domain 2 (hMBD2) nucleic acid and amino acid sequence variants and the use of these variants as a simple and sensitive technology for the detection of CpG methylation in DNA. This hMBD2 variants of the invention bind methylated DNA. In particular, the hMBD2 variants of the invention recognize and/or bind DNA comprising a single methylated CpG site, with high affinity.

In one embodiment, the present invention provides isolated nucleic acids of DNA, RNA, and analogs and/or chimeras thereof, comprising a polynucleotide, wherein said polynucleotide encodes a variant human methyl binding domain 2 (hMBD2) polypeptide.

In a one embodiment, the invention provides a variant hMBD2 polypeptide comprising a sequence selected from:

(SEQ ID NO: 7)
ESGKRMDCPALPPGWKKEVVIRKSGLSAGKSDVYYFSPSGKKFRSKPQLA
RYLGNTVDLSSFDYRTGKM;
(SEQ ID NO: 8)
ESGKRMDCPALPPGWKREEVIRKSGLSAGKSDVYYFSPSGKKFRSKPQLA
RYLGNTVDLSSFDFRTGKM;
(SEQ ID NO: 9)
ESGKRMDCPALPPGWKREEVIRKSGLSAGKSDVYYFSPSGKKFRSKPQLA
RYLGNTVDLSSFDYRTGKM;
(SEQ ID NO: 10)
ESGKRMDCPALPPGWKKEEVIRKSGLSAGKIDVYYFSPSGKKIRSKPQLA
RYLGNTVDLSSFDFRTGKM;
(SEQ ID NO: 11)
ESGKRMDCPALPPGWKREEVIRKSGLSAGKRDVYYFSPSGKKFRSKPQLA
RYLGNTVDLSSFDFRTGKM;
(SEQ ID NO: 12)
ESGKRMDCPALPPGWKREEVIRKSGLSAGKIDVYYFSPSGKKFRSKPQLA
RYLGNTVDLSSFDFRTGKM;
(SEQ ID NO: 13)
ESGKRTDCPALPPGWKREEVIRKSGLSAGKSDVYYFSPSGKKFRSKPQLA
RYLGNTVDLSSFDFRTGKM;
(SEQ ID NO: 14)
ESGKRMDCPALPPGWKREEVIRKSGRSAGKIDVYYFSPSGKKIRSKPQLA
RYLGNTVDLSSFDYRTGKM;
(SEQ ID NO: 15)
ESGKRMDCPALPPGWKREEVIRKSGLSAGKSDVYYFSPSGKKFRSKRQLA
RYLGNTVDLSSFDYRTGKM;
(SEQ ID NO: 22)
ESGKRMDCPALPPGWKREEVIRKSGLSAGKIDVYYFSPSGKKIRSKPQLA
RYLGNTVDLSSFDFRTCKM;
(SEQ ID NO: 23)
ESGKRMDCPALPPGWKREEVIRKSGLSAGKSDVYYFSPSGKKIRSKPQLA
RYLGNSVDLSSFDYRTGKM;
(SEQ ID NO: 24)
ESGKRMDCPALPPGWKREEVIRKSGLSAGKSDVYYYSPSGKKFRSKPQLA
RYLGNTVDLSSFDYRTGKM;
(SEQ ID NO: 25)
ESGKRMDCPALPPGWKREEVIRKSGLSAGKSDVYYFSPSGKKIRSKPQLA
RYLGNTVDLSSFDYRTGKM;
or
(SEQ ID NO: 26)
ESGKRMDCPALPPGWKREEVIRKSGLSAGKIDVYYFSPSGKKIRSKPQLA
RYLGNTVDLSSFDYRTGKM.

In one embodiment, the invention provides a polynucleotide which encodes a variant hMBD2 polypeptide of the invention comprising a sequence selected from:

(SEQ ID NO: 1)
GAAAGCGGCAAACGCATGGATTGCCCGGCGCTGCCGCCGGGTTGGAAAAG
AGAAGAAGTGATTCGTAAAAGCGGCCGGAGCGCGGGCAAAATCGATGTGT
ATTATTTTAGCCCGAGCGGCAAAAAAATTCGTAGCAAACCGCAGCTGGCG
CGTTATCTGGGCAACACCGTGGATCTGAGCAGCTTTGATTATCGTACCGG
CAAAATG;
(SEQ ID NO: 27)
GAAAGCGGCAAACGCATGGATTGCCCGGCGCTGCCGCCGGGTTGGAAAAG
AGAAGAAGTGATTCGTAAAAGCGGCCTGAGCGCGGGCAAAAGCGATGTGT
ATTATTTTAGCCCGAGCGGCAAAAAATTTCGTAGCAAACCGCAGCTGGCG
CGTTATCTGGGCAACACCGTGGATCTGAGCAGCTTTGATTTTCGTACCGG
CAAAATG;
(SEQ ID NO: 28)
GAAAGCGGCAAACGCATGGATTGCCCGGCGCTGCCGCCGGGTTGGAAAAG
AGAAGAAGTGATTCGTAAAAGCGGCCTGAGCGCGGGCAAAAGCGATGTGT
ATTATTTTAGCCCGAGCGGCAAAAAATTTCGTAGCAAACCGCAGCTGGCG
CGTTATCTGGGCAACACCGTGGATCTGAGCAGCTTTGATTATCGTACCGG
CAAAATG;
(SEQ ID NO: 29)
GAAAGCGGCAAACGCATGGATTGCCCGGCGCTGCCGCCGGGTTGGAAAAG
AGAAGAAGTGATTCGTAAAAGCGGCCTGAGCGCGGGCAAAATCGATGTGT
ATTATTTTAGCCCGAGCGGCAAAAAATTTCGTAGCAAACCGCAGCTGGCG
CGTTATCTGGGCAACACCGTGGATCTGAGCAGCTTTGATTTTCGTACCGG
CAAAATG;
(SEQ ID NO: 30)
GAAAGCGGCAAACGCACGGATTGCCCGGCGCTGCCGCCGGGTTGGAAAAG
AGAAGAAGTGATTCGTAAAAGCGGCCTGAGCGCGGGCAAAAGCGATGTGT
ATTATTTTAGCCCGAGCGGCAAAAAATTTCGTAGCAAACCGCAGCTGGCG
CGTTATCTGGGCAACACCGTGGATCTGAGCAGCTTTGATTTTCGTACCGG
CAAAATG;
(SEQ ID NO: 31)
GAAAGCGGCAAACGCATGGATTGCCCGGCGCTGCCGCCGGGTTGGAAAAG
AGAAGAAGTGATTCGTAAAAGCGGCCTGAGCGCGGGCAAAAGCGATGTGT
ATTATTTTAGCCCGAGCGGCAAAAAATTTCGTAGCAAACGGCAGCTGGCG
CGTTATCTGGGCAACACCGTGGATCTGAGCAGCTTTGATTATCGTACCGG
CAAAATG;
(SEQ ID NO: 32)
GAAAGCGGCAAACGCATGGATTGCCCGGCGCTGCCGCCGGGTTGGAAAAG
AGAAGAAGTGATTCGTAAAAGCGGCCTGAGCGCGGGCAAAATCGATGTGT
ATTATTTTAGCCCGAGCGGCAAAAAAATTCGTAGCAAACCGCAGCTGGCG
CGTTATCTGGGCAACACCGTGGATCTGAGCAGCTTTGATTTTCGTACCTG
CAAAATG;
(SEQ ID NO: 33)
GAAAGCGGCAAACGCATGGATTGCCCGGCGCTGCCGCCGGGTTGGAAAAG
AGAAGAAGTGATTCGTAAAAGCGGCCTGAGCGCGGGCAAAAGCGATGTGT
ATTATTTTAGCCCGAGCGGCAAAAAAATTCGTAGCAAACCGCAGCTGGCG
CGTTATCTGGGCAACTCCGTGGATCTGAGCAGCTTTGATTATCGTACCGG
CAAAATG;
(SEQ ID NO: 34)
GAAAGCGGCAAACGCATGGATTGCCCGGCGCTGCCGCCGGGTTGGAAAAG
AGAAGAAGTGATTCGTAAAAGCGGCCTGAGCGCGGGCAAAAGCGATGTGT
ATTATTATAGCCCGAGCGGCAAAAAATTTCGTAGCAAACCGCAGCTGGCG
CGTTATCTGGGCAACACCGTGGATCTGAGCAGCTTTGATTATCGTACCGG
CAAAATG;
(SEQ ID NO: 35)
GAAAGCGGCAAACGCATGGATTGCCCGGCGCTGCCGCCGGGTTGGAAAAG
AGAAGAAGTGATCCGTAAAAGCGGCCTGAGCGCGGGCAAAAGCGATGTGT
ATTATTTTAGCCCGAGCGGCAAAAAAATTCGTAGCAAACCGCAGCTGGCG
CGTTATCTGGGCAACACCGTGGATCTGAGCAGCTTTGATTATCGTACCGG
CAAAATG;
or
(SEQ ID NO. 36)
GAAAGCGGCAAACGCATGGATTGCCCGGCGCTGCCGCCGGGTTGGAAAAG
AGAAGAAGTGATTCGTAAAAGCGGCCTGAGCGCGGGCAAAATCGATGTGT
ATTATTTTAGCCCGAGCGGCAAAAAAATTCGTAGCAAACCGCAGCTGGCG
CGTTATCTGGGCAACACCGTGGATCTGAGCAGCTTTGATTATCGTACCGG
CAAAATG.

All nucleotide sequences are 5′ to 3′ unless otherwise noted.

The present invention further provides conservatively modified variants of the polynucleotide of SEQ ID NO: 1, SEQ ID NO: 27; SEQ ID NO: 28; SEQ ID NO: 29; SEQ ID NO; 30; SEQ ID NO: 31; SEQ ID NO: 32; SEQ ID NO: 33; SEQ ID NO: 34; SEQ ID NO; 35; or SEQ ID NO: 36. It is known in the art that the degeneracy of the genetic code allows for a plurality of polynucleotides to encode for the identical amino acid sequence. These “silent variations” can be used to encode the polypeptide of SEQ ID NO: 7, SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 22; SEQ ID NO: 23; SEQ ID NO: 24; SEQ ID NO: 25; or SEQ ID NO: 26.

In one embodiment, the present invention provides a variant hMBD2 polypeptide of SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO; 14; SEQ ID NO: 15; SEQ ID NO: 22; SEQ ID NO: 23; SEQ ID NO: 24; SEQ ID NO: 25; or SEQ ID NO: 26. In another embodiment, the present invention provides a conservatively modified variant of the polypeptide of SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 22; SEQ ID NO: 23; SEQ ID NO: 24; SEQ ID NO: 25; or SEQ ID NO: 26. In one embodiment, such a modified polypeptide binds a DNA sequence having a single methylated CpG site with a dissociation constant (Kd) of greater than or equal to 3.1±1.0 nM. The dissociation constant can be determined by one skilled in the art using routine methods, for example, as those described herein.

In a one embodiment, the invention provides a variant hMBD2 polypeptide comprising the sequence ESGKRMDCPALPPGWKKEVVIRKSGLSAGKSDVYYFSPSGKKFRSKPQLARYLGNTVD LSSFDYRTGKM (SEQ ID NO; 7).

In one embodiment, the invention provides a polynucleotide which encodes the variant hMBD2 polypeptide of SEQ ID NO; 7. The present invention further provides conservatively modified variants of the polynucleotide which encodes the variant hMBD2 polypeptide of SEQ ID NO: 7. It is known in the art that the degeneracy of the genetic code allows for a plurality of polynucleotides to encode for the identical amino acid sequence. These “silent variations” can be used to encode the polypeptide of SEQ ID NO: 7.

In one embodiment, the present invention provides a variant hMBD2 polypeptide of SEQ ID NO: 7. In another embodiment, the present invention provides a conservatively modified variant of the polypeptide of SEQ ID NO: 7. In one embodiment, such a modified polypeptide binds a DNA sequence having a single methylated CpG site with a dissociation constant (Kd) of greater than or equal to 3.1±1.0 nM. The dissociation constant can be determined by one skilled in the art using routine methods, for example, as those described herein.

In a one embodiment, the invention provides a variant hMBD2 polypeptide comprising the sequence ESGKRMDCPALPPGWKREEVIRKSGLSAGKSDVYYFSPSGKKFRSKPQLARYLGNTVDL SSFDFRTGKM (SEQ ID NO: 8).

In one embodiment, the polynucleotide which encodes the variant hMBD2 polypeptide of SEQ ID NO: 8 comprises the sequence 5′-GAAAGCGGCAAACGCATGGATTGCCCGGCGCTGCCGCCGGGTTGGAAAAGAGAAG AAGTGATTCGTAAAAGCGGCCTGAGCGCGGGCAAAAGCGATGTGTATTATTTTAGC CCGAGCGGCAAAAAATTTCGTAGCAAACCGCAGCTGGCGCGTTATCTGGGCAACAC CGTGGATCTGAGCAGCTTTGATTTTCGTACCGGCAAAATG-3′ (SEQ ID NO: 27). The present invention further provides conservatively modified variants of the polynucleotide of SEQ ID NO: 27. It is known in the art that the degeneracy of the genetic code allows for a plurality of polynucleotides to encode for the identical amino acid sequence. These “silent variations” can be used to encode the polypeptide of SEQ ID NO: 8.

In one embodiment, the present invention provides a variant hMBD2 polypeptide of SEQ ID NO: 8. In another embodiment, the present invention provides a conservatively modified variant of the polypeptide of SEQ ID NO: 8. In one embodiment, such a modified polypeptide binds a DNA sequence having a single methylated CpG site with a dissociation constant (Kd) of greater than or equal to 3.1±1.0 nM. The dissociation constant can be determined by one skilled in the art using routine methods, for example, as those described herein.

In a one embodiment, the invention provides a variant hMBD2 polypeptide comprising the sequence ESGKRMDCPALPPGWKREEVIRKSGLSAGKSDVYYFSPSGKKFRSKPQLARYLGNTVDL SSFDYTGKM (SEQ ID NO: 9).

In one embodiment, the polynucleotide which encodes the variant hMBD2 polypeptide of SEQ ID NO: 9 comprises the sequence 5′-GAAAGCGGCAAACGCATGGATTGCCCGGCGCTGCCGCCGGGTTGGAAAAGAGAAG AAGTGATTCGTAAAAGCGGCCTGAGCGCGGGCAAAAGCGATGTGTATTATTTTAGC CCGAGCGGCAAAAAATTTCGTAGCAAACCGCAGCTGGCGCGTTATCTGGGCAACAC CGTGGATCTGAGCAGCTTTGATTATCGTACCGGCAAAATG-3′ (SEQ ID NO: 28). The present invention further provides conservatively modified variants of the polynucleotide of SEQ ID NO: 28. It is known in the art that the degeneracy of the genetic code allows for a plurality of polynucleotides to encode for the identical amino acid sequence. These “silent variations” can be used to encode the polypeptide of SEQ ID NO: 9.

In one embodiment, the present invention provides a variant hMBD2 polypeptide of SEQ ID NO: 9. In another embodiment, the present invention provides a conservatively modified variant of the polypeptide of SEQ ID NO: 9. In one embodiment, such a modified polypeptide binds a DNA sequence having a single methylated CpG site with a dissociation constant (Kd) of greater than or equal to 3.1±1.0 nM. The dissociation constant can be determined by one skilled in the art using routine methods, for example, as those described herein.

In a one embodiment, the invention provides a variant hMBD2 polypeptide comprising the sequence ESGKRMDCPALPPGWKKEEVIRKSGLSAGKIDVYYFSPSGKIRSKPQLARYLGNTVDL SSFDFRTGKM (SEQ ID NO: 10).

In one embodiment, the invention provides the polynucleotide which encodes the variant hMBD2 polypeptide of SEQ ID NO: 10. The present invention further provides conservatively modified variants of the polynucleotide which encodes the variant hMBD2 polypeptide of SEQ ID NO: 10. It is known in the art that the degeneracy of the genetic code allows for a plurality of polynucleotides to encode for the identical amino acid sequence. These “silent variations” can be used to encode the polypeptide of SEQ ID NO: 10.

In one embodiment, the present invention provides a variant hMBD2 polypeptide of SEQ ID NO: 10. In another embodiment, the present invention provides a conservatively modified variant of the polypeptide of SEQ ID NO: 10. In one embodiment, such a modified polypeptide binds a DNA sequence having a single methylated CpG site with a dissociation constant (Kd) of greater than or equal to 3.1±1.0 nM. The dissociation constant can be determined by one skilled in the art using routine methods, for example, as those described herein.

In a one embodiment, the invention provides a variant hMBD2 polypeptide comprising the sequence ESGKRMDCPALPPGWKREEVIRKSGLSAGKRDVYYFSPSGKKFRSKPQLARYLGNTVD LSSFDFRTGKM (SEQ ID NO: 11).

In one embodiment, the invention provides the polynucleotide which encodes the variant hMBD2 polypeptide of SEQ ID NO: 11. The present invention further provides conservatively modified variants of the polynucleotide which encodes the variant hMBD2 polypeptide of SEQ ID NO: 11. It is known in the art that the degeneracy of the genetic code allows for a plurality of polynucleotides to encode for the identical amino acid sequence. These “silent variations” can be used to encode the polypeptide of SEQ ID NO: 11.

In one embodiment, the present invention provides a variant hMBD2 polypeptide of SEQ ID NO: 11. In another embodiment, the present invention provides a conservatively modified variant of the polypeptide of SEQ ID NO: 11. In one embodiment, such a modified polypeptide binds a DNA sequence having a single methylated CpG site with a dissociation constant (Kd) of greater than or equal to 3.1±1.0 nM. The dissociation constant can be determined by one skilled in the art using routine methods, for example, as those described herein.

In a one embodiment, the invention provides a variant hMBD2 polypeptide comprising the sequence ESGKRMDCPALPPGWKREEVIRKSGLSAGKIDVYYFSPSGKKFRSKPQLARYLGNTVDL SSFDFRTGKM (SEQ ID NO: 12).

In one embodiment, the polynucleotide which encodes the variant hMBD2 polypeptide of SEQ ID NO: 12 comprises the sequence 5′-GAAAGCGGCAAACGCATGGATTGCCCGGCGCTGCCGCCGGGTTGGAAAAGAGAAG AAGTGATTCGTAAAAGCGGCCTGAGCGCGGGCAAAATCGATGTGTATTATTTTAGCC CGAGCGGCAAAAAATTTCGTAGCAAACCGCAGCTGGCGCGTTATCTGGGCAACACC GTGGATCTGAGCAGCTTTGATTTTCGTACCGGCAAAATG-3′ (SEQ ID NO: 29). The present invention further provides conservatively modified variants of the polynucleotide of SEQ ID NO: 29. It is known in the art that the degeneracy of the genetic code allows for a plurality of polynucleotides to encode for the identical amino acid sequence. These “silent variations” can be used to encode the polypeptide of SEQ ID NO: 12.

In one embodiment, the present invention provides a variant hMBD2 polypeptide of SEQ ID NO: 12. In another embodiment, the present invention provides a conservatively modified variant of the polypeptide of SEQ ID NO: 12. In one embodiment, such a modified polypeptide binds a DNA sequence having a single methylated CpG site with a dissociation constant (Kd) of greater than or equal to 3.1±1.0 nM. The dissociation constant can be determined by one skilled in the art using routine methods, for example, as those described herein.

In a one embodiment, the invention provides a variant hMBD2 polypeptide comprising the sequence ESGKRTDCPALPPGWKREEVIRKSGLSAGKSDVYYFSPSGKKFRSKPQLARYLGNTVDL SSFDFRTGKM (SEQ ID NO: 13).

In one embodiment, the polynucleotide which encodes the variant hMBD2 polypeptide of SEQ ID NO: 13 comprises the sequence 5′-GAAAGCGGCAAACGCACGGATTGCCCGGCGCTGCCGCCGGGTTGGAAAAGAGAAG AAGTGATTCGTAAAAGCGGCCTGAGCGCGGGCAAAAGCGATGTGTATTATTTTAGC CCGAGCGGCAAAAAATTTCGTAGCAAACCGCAGCTGGCGCGTTATCTGGGCAACAC CGTGGATCTGAGCAGCTTTGATTTTCGTACCGGCAAAATG-3′ (SEQ ID NO: 30). The present invention further provides conservatively modified variants of the polynucleotide of SEQ ID NO: 30. It is known in the art that the degeneracy of the genetic code allows for a plurality of polynucleotides to encode for the identical amino acid sequence. These “silent variations” can be used to encode the polypeptide of SEQ ID NO: 13.

In one embodiment, the present invention provides a variant hMBD2 polypeptide of SEQ ID NO: 13. In another embodiment, the present invention provides a conservatively modified variant of the polypeptide of SEQ ID NO: 13. In one embodiment, such a modified polypeptide binds a DNA sequence having a single methylated CpG site with a dissociation constant (Kd) of greater than or equal to 3.1±1.0 nM. The dissociation constant can be determined by one skilled in the art using routine methods, for example, as those described herein.

In a one embodiment, the invention provides a variant hMBD2 polypeptide comprising the sequence ESGKRMDCPALPPGWKREEVIRKSGRSAGKIDVYYFSPSGKKIRSKPQLARYLGNTVDL SSFDYRTGKM (SEQ ID NO: 14).

In one embodiment, the polynucleotide which encodes the variant hMBD2 polypeptide of SEQ ID NO: 14 comprises the sequence 5′-GAAAGCGGCAAACGCATGGATTGCCCGGCGCTGCCGCCGGGTTGGAAAAGAGAAG AAGTGATTCGTAAAAGCGGCCGGAGCGCGGGCAAAATCGATGTGTATTATTTTAGC CCGAGCGGCAAAAAAATTCGTAGCAAACCGCAGCTGGCGCGTTATCTGGGCAACAC CGTGGATCTGAGCAGCTTTGATTATCGTACCGGCAAAATG-3′ (SEQ ID NO: 1). The present invention further provides conservatively modified variants of the polynucleotide of SEQ ID NO: 1. It is known in the art that the degeneracy of the genetic code allows for a plurality of polynucleotides to encode for the identical amino acid sequence. These “silent variations” can be used to encode the polypeptide of SEQ ID NO: 14.

In one embodiment, the present invention provides a variant hMBD2 polypeptide of SEQ ID NO: 14. In another embodiment, the present invention provides a conservatively modified variant of the polypeptide of SEQ ID NO: 14. In one embodiment, such a modified polypeptide binds a DNA sequence having a single methylated CpG site with a dissociation constant (Kd) of greater than or equal to 3.1±1.0 nM. The dissociation constant can be determined by one skilled in the art using routine methods, for example, as those described herein.

In a one embodiment, the invention provides a variant hMBD2 polypeptide comprising the sequence ESGKRMDCPALPPGWKREEVIRKSGLSAGKSDVYYFSPSGKKFRSKRQLARYLGNTVD LSSFDYRTGKM (SEQ ID NO: 15).

In one embodiment, the polynucleotide which encodes the variant hMBD2 polypeptide of SEQ ID NO: 15 comprises the sequence 5′-GAAAGCGGCAAACGCATGGATTGCCCGGCGCTGCCGCCGGGTTGGAAAAGAGAAG AAGTGATTCGTAAAAGCGGCCTGAGCGCGGGCAAAAGCGATGTGTATTATTTTAGC CCGAGCGGCAAAAAATTTCGTAGCAAACGGCAGCTGGCGCGTTATCTGGGCAACAC CGTGGATCTGAGCAGCTTTGATTATCGTACCGGCAAAATG-3′ (SEQ ID NO: 31). The present invention further provides conservatively modified variants of the polynucleotide of SEQ ID NO: 31. It is known in the art that the degeneracy of the genetic code allows for a plurality of polynucleotides to encode for the identical amino acid sequence. These “silent variations” can be used to encode the polypeptide of SEQ ID NO: 15.

In one embodiment, the present invention provides a variant hMBD2 polypeptide of SEQ ID NO: 15. In another embodiment, the present invention provides a conservatively modified variant of the polypeptide of SEQ ID NO: 15. In one embodiment, such a modified polypeptide binds a DNA sequence having a single methylated CpG site with a dissociation constant (Kd) of greater than or equal to 3.1±1.0 nM. The dissociation constant can be determined by one skilled in the art using routine methods, for example, as those described herein.

In one embodiment, the invention provides a variant hMBD2 polypeptide comprising the sequence ESGKRMDCPALPPGWKREEVIRKSGLSAGKIDVYYFSPSGKKIRSKPQLARYLGNTVDL SSFDFRTCKM (SEQ ID NO: 22).

In one embodiment, the polynucleotide which encodes the variant hMBD2 polypeptide of SEQ ID NO: 22 comprises the sequence 5′-GAAAGCGGCAAACGCATGGATTGCCCGGCGCTGCCGCCGGGTTGGAAAAGAGAAG AAGTGATTCGTAAAAGCGGCCTGAGCGCGGGCAAAATCGATGTGTATTATTTAGCC CGAGCGGCAAAAAAATTCGTAGCAAACCGCAGCTGGCGCGTTATCTGGGCAACACC GTGGATCTGAGCAGCTTTGATTTTCGTACCTGCAAAATG-3′ (SEQ ID NO; 32). The present invention further provides conservatively modified variants of the polynucleotide of SEQ ID NO: 32. It is known in the art that the degeneracy of the genetic code allows for a plurality of polynucleotides to encode for the identical amino acid sequence. These “silent variations” can be used to encode the polypeptide of SEQ ID NO: 22.

In one embodiment, the present invention provides a variant hMBD2 polypeptide of SEQ ID NO: 22. In another embodiment, the present invention provides a conservatively modified variant of the polypeptide of SEQ ID NO: 22. In one embodiment, such a modified polypeptide binds a DNA sequence having a single methylated CpG site with a dissociation constant (Kd) of greater than or equal to 3.1±1.0 nM. The dissociation constant can be determined by one skilled in the art using routine methods, for example, as those described herein.

In one embodiment, the invention provides a variant hMBD2 polypeptide comprising the sequence ESGKRMDCPALPPGWKREEVIRKSGLSAGKSDVYYFSPSGKKIRSKPQLARYLGNSVDL SSFDYRTGKM (SEQ ID NO: 23).

In one embodiment, the polynucleotide which encodes the variant hMBD2 polypeptide of SEQ ID NO: 23 comprises the sequence 5′-GAAAGCGGCAAACGCATGGATTGCCCGGCGCTGCCGCCGGGTTGGAAAAGAGAAG AAGTGATTCGTAAAAGCGGCCTGAGCGCGGGCAAAAGCGATGTGTATTATTTTAGC CCGAGCGGCAAAAAAATTCGTAGCAAACCGCAGCTGGCGCGTTATCTGGGCAACTC CGTGGATCTGAGCAGCTTTGATTATCGTACCGGCAAAATG-3′ (SEQ ID NO: 33). The present invention further provides conservatively modified variants of the polynucleotide of SEQ ID NO: 33. It is known in the art that the degeneracy of the genetic code allows for a plurality of polynucleotides to encode for the identical amino acid sequence. These “silent variations” can be used to encode the polypeptide of SEQ ID NO: 23.

In one embodiment, the present invention provides a variant hMBD2 polypeptide of SEQ ID NO: 23. In another embodiment, the present invention provides a conservatively modified variant of the polypeptide of SEQ ID NO: 23. In one embodiment, such a modified polypeptide binds a DNA sequence having a single methylated CpG site with a dissociation constant (Kd) of greater than or equal to 3.1±1.0 nM. The dissociation constant can be determined by one skilled in the art using routine methods, for example, as those described herein.

In one embodiment, the invention provides a variant hMBD2 polypeptide comprising the sequence ESGKRMDCPALPPGWKREEVIRKSGLSAGKSDVYYYSPSGKKFRSKPQLARYLGNTVD LSSFDYRTGKM (SEQ ID NO: 24).

In one embodiment, the polynucleotide which encodes the variant hMBD2 polypeptide of SEQ ID NO: 24 comprises the sequence 5′-GAAAGCGGCAAACGCATGGATTGCCCGGCGCTGCCGCCGGGTTGGAAAAGAGAAG AAGTGATTCGTAAAAGCGGCCTGAGCGCGGGCAAAAGCGATGTGTATTATTATAGC CCGAGCGGCAAAAAATTTCGTAGCAAACCGCAGCTGGCGCGTTATCTGGGCAACAC CGTGGATCTGAGCAGCTTTGATTATCGTACCGGCAAAATG-3′ (SEQ ID NO: 34). The present invention further provides conservatively modified variants of the polynucleotide of SEQ ID NO: 34. It is known in the art that the degeneracy of the genetic code allows for a plurality of polynucleotides to encode for the identical amino acid sequence. These “silent variations” can be used to encode the polypeptide of SEQ ID NO: 24.

In one embodiment, the present invention provides a variant hMBD2 polypeptide of SEQ ID NO: 24. In another embodiment, the present invention provides a conservatively modified variant of the polypeptide of SEQ ID NO: 24. In one embodiment, such a modified polypeptide binds a DNA sequence having a single methylated CpG site with a dissociation constant (Kd) of greater than or equal to 3.1±1.0 nM. The dissociation constant can be determined by one skilled in the art using routine methods, for example, as those described herein.

In a one embodiment, the invention provides a variant hMBD2 polypeptide comprising the sequence ESGKRMDCPALPPGWKREEVIRKSGLSAGKSDVYYFSPSGKKIRSKPQLARYLGNTVDL SSFDYRTGKM (SEQ ID NO: 25).

In one embodiment, the polynucleotide which encodes the variant hMBD2 polypeptide of SEQ ID NO: 25 comprises the sequence 5′-GAAAGCGGCAAACGCATGGATTGCCCGGCGCTGCCGCCGGGTTGGAAAAGAGAAG AAGTGATCCGTAAAAGCGGCCTGAGCGCGGGCAAAAGCGATGTGTATTATTTTAGC CCGAGCGGCAAAAAAATTCGTAGCAAACCGCAGCTGGCGCGTTATCTGGGCAACAC CGTGGATCTGAGCAGCTTTGATTATCGTACCGGCAAAATG-3′ (SEQ ID NO: 35). The present invention further provides conservatively modified variants of the polynucleotide of SEQ ID NO: 35. It is known in the art that the degeneracy of the genetic code allows for a plurality of polynucleotides to encode for the identical amino acid sequence. These “silent variations” can be used to encode the polypeptide of SEQ ID NO: 25.

In one embodiment, the present invention provides a variant hMBD2 polypeptide of SEQ ID NO: 25. In another embodiment, the present invention provides a conservatively modified variant of the polypeptide of SEQ ID NO: 25. In one embodiment, such a modified polypeptide binds a DNA sequence having a single methylated CpG site with a dissociation constant (Kd) of greater than or equal to 3.1±1.0 nM. The dissociation constant can be determined by one skilled in the art using routine methods, for example, as those described herein.

In one embodiment, the invention provides a variant hMBD2 polypeptide comprising the sequence ESGKRMDCPALPPGWKREEVIRKSGLSAGKIDVYYFSPSGKKIRSKPQLARYLGNTVDL SSFDYRTGKM (SEQ ID NO: 26).

In one embodiment, the polynucleotide which encodes the variant hMBD2 polypeptide of SEQ ID NO: 26 comprises the sequence 5′-GAAAGCGGCAAACGCATGGATTGCCCGGCGCTGCCGCCGGGTTGGAAAAGAGAAG AAGTGATTCGTAAAAGCGGCCTGAGCGCGGGCAAAATCGATGTGTATTAITTTTAGCC CGAGCGGCAAAAAAATTCGTAGCAAACCGCAGCTGGCGCGTTATCTGGGCAACACC GTGGATCTGAGCAGCTTTGATTATCGTACCGGCAAAATG-3′ (SEQ ID NO: 36). The present invention further provides conservatively modified variants of the polynucleotide of SEQ ID NO: 36. It is known in the art that the degeneracy of the genetic code allows for a plurality of polynucleotides to encode for the identical amino acid sequence. These “silent variations” can be used to encode the polypeptide of SEQ ID NO: 26.

In one embodiment, the present invention provides a variant hMBD2 polypeptide of SEQ ID NO: 26. In another embodiment, the present invention provides a conservatively modified variant of the polypeptide of SEQ ID NO: 26. In one embodiment, such a modified polypeptide binds a DNA sequence having a single methylated CpG site with a dissociation constant (Kd) of greater than or equal to 3.1±1.0 nM. The dissociation constant can be determined by one skilled in the art using routine methods, for example, as those described herein.

In one embodiment, the polynucleotide which encodes the variant hMBD2 polypeptide of SEQ ID NO: 14 comprises the sequence (GAAAGCGGCAAACGCATGGATTGCCCGGCGCTGCCGCCGGGTTGGAAAAGAGAAG AAGTGATTCGTAAAAGCGGCCGGAGCGCGGGCAAAATCGATGTGTATTATTTTAGC CCGAGCGGCAAAAAAATTCGTAGCAAACCGCAGCTGGCGCGTTATCTGGGCAACAC CGTGGATCTGAGCAGCTTTGATTATCGTACCGGCAAAATG/SEQ ID NO: 1). The present invention further provides conservatively modified variants of the polynucleotide of SEQ ID NO: 1. It is known in the art that the degeneracy of the genetic code allows for a plurality of polynucleotides to encode for the identical amino acid sequence. These “silent variations” can be used to encode the polypeptide of SEQ ID NO: 14.

In one embodiment, the present invention provides a variant hMBD2 polypeptide of SEQ ID NO: 14. In another embodiment, the present invention provides a conservatively modified variant of the polypeptide of SEQ ID NO: 14 provided that such a modified polypeptide binds a DNA sequence having a single methylated CpG site with at a binding affinity (Kd) of at least 3.1±1.0 nM. The dissociation constant/binding affinity can be determined by one skilled in the art using routine methods, for example, as those described herein.

The present invention further provides fusion proteins that bind to methylated CpG DNA. Such fusion proteins comprise a variant hMBD2 polypeptide of the invention and a reporter protein. In one embodiment, the variant hMBD2 polypeptide comprises a sequence selected from SEQ ID NO: 7; SEQ ID NO: 8. SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 22; SEQ ID NO: 23, SEQ ID NO: 24; SEQ ID NO: 25; or SEQ ID NO: 26.

The present invention further provides fusion proteins that bind to methylated CpG DNA. Such fusion proteins comprise a variant hMBD2 polypeptide of the invention and a reporter protein. In one embodiment, the variant hMBD2 polypeptide comprises SEQ ID NO: 14.

The present invention further provides fusion proteins that bind to methylated CpG DNA. Such fusion proteins comprise a variant hMBD2 polypeptide of the invention and a reporter protein. In one embodiment, the variant hMBD2 polypeptide comprises SEQ ID NO: 23.

Also provided are polynucleotides encoding the fusion polypeptides of the invention. In some embodiments, the nucleic acid molecule of the present invention is part of a vector. The present invention relates in another embodiment to a vector comprising the nucleic acid molecule of this invention. Such a vector may be, e.g., a plasmid, cosmid, virus, bacteriophage or another vector used e.g. conventionally in genetic engineering, and may comprise further genes such as marker or reporter genes which allow for the selection and/or replication and/or detection of said vector in a suitable host cell and under suitable conditions. In one embodiment, said vector is an expression vector, in which the nucleic acid molecule of the present invention is operatively linked to an expression control sequence(s) (e.g., a promotor) allowing expression in prokaryotic or eukaryotic host cells as described herein.

These variant hMBD2 sequences can be incorporated into vectors as multimerized constructs with a reporter (e.g., an enhanced green fluorescent protein (eGFP)) tag. For example, single peptides with 2-500, preferably 2-250, preferably 2-100, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 copies of the variant hMBD2 polypeptides according to the invention and a C-terminal reporter (e.g., eGFP) tag can be prepared. In one embodiment, the variant hMBD2 polypeptide comprises SEQ ID NO: 14. In one embodiment, the variant hMBD2 polypeptide comprises SEQ ID NO: 23.

The present invention relates also to an in vitro method for detecting methylated DNA comprising contacting a sample comprising methylated and/or unmethylated DNA with the polypeptide of the present invention; and detecting the binding of said polypeptide to methylated DNA.

In one embodiment, said in vitro method is reverse South-Western blotting, immune precipitation, affinity purification of methylated DNA or Methyl-CpG-immunoprecipitation (MCIp). However, said in vitro method is not limited thereto, but could basically be any procedure in which the polypeptide of the present invention is linked to a solid matrix, for example, a matrix such as sepharose, agarose, capillaries, vessel walls, as is also described herein in connection with the diagnostic composition of the present invention.

In another embodiment, the aforementioned in vitro methods further comprise as step (c) analyzing the methylated DNA, for example, by sequencing, Southern Blot, restriction enzyme digestion, bisulfite sequencing, pyrosequencing or PCR. Yet, analyzing methylated DNA which has been isolated, enriched, purified and/or detected by using the polypeptide of the present invention is not limited to the aforementioned methods, but encompasses all methods known in the art for analyzing methylated DNA, e.g., RDA, microarrays and the like.

In some embodiments, detection methods comprise, but are not limited to, autoradiography, fluorescence microscopy, direct and indirect enzymatic reactions, etc. The use of a fluorescent tag (e.g., eGFP and HA tags) allow the variant hMBD2 proteins of the invention to transduce binding to methylated DNA to a directly observable signal which reduces assay complexity, reduces time, and eliminates the need for DNA sequencing.

Accordingly, in one embodiment the composition according to the invention is a diagnostic composition, optionally further comprising suitable means for detection.

A further embodiment of the present invention is the use of the polypeptide of the present invention for the detection of methylated DNA.

In addition, the nucleic acid molecules, the polypeptide, or the vector, of the present invention are used for the preparation of a diagnostic composition for detecting methylated DNA.

Additionally, the present invention provides a kit comprising the nucleic acid molecule, the vector, or the polypeptide of the present invention.

Advantageously, the kit of the present invention further comprises, optionally (a) reaction buffer(s), storage solutions and/or remaining reagents or materials required for the conduct of scientific or diagnostic assays or the like. Furthermore, parts of the kit of the invention can be packaged individually in vials or bottles or in combination in containers or multicontainer units.

The kit of the present invention may be advantageously used, inter alia, for carrying out the method for isolating, enriching, purifying and/or detecting methylated DNA as described herein and/or it could be employed in a variety of applications referred herein, e.g., as diagnostic kits, as research tools or therapeutic tools. Additionally, the kit of the invention may contain means for detection suitable for scientific, medical and/or diagnostic purposes. The manufacture of the kits follows preferably standard procedures which are known to the person skilled in the art.

Instructions for use may be included in the kit. “Instructions for use” typically include a tangible expression describing the technique to be employed in using the components of the kit to effect a desired outcome, such as to detect DNA methylation.

### EXAMPLES

### Example 1

### Displaying MBD Proteins on the Surface of S. cerevisiae Yeast Cells

The cDNA encoding the hMBD2 gene (AAs 145-213/SEQ ID NO: 16) was PCR amplified from the pMal-c2X-MBD2 construct (Porter et al., 2007) from Indraneel Ghosh (University of Arizona). The forward 5′-TAC AGC TAG CGA AAG CGG CAA ACG-3′ (SEQ ID NO: 17), and reverse 5′-GAC AGG ATC CCA TTT TGC CGG TAC GA-3′ (SEQ ID NO: 18) primer pair was designed to append flanking 5′ NheI and 3′ BamHI restriction sites. The PCR reaction was carried out as described above. The thermocycling profile was as follows: initial denaturation at 98° C. for 30 sec followed by 30 cycles of denaturation at 98° C. for 10 sec, annealing at 60° C. for 30 sec, extension at 72° C. for 30 sec, and a final extension at 72° C. for 10 min. All other steps were performed as described above.

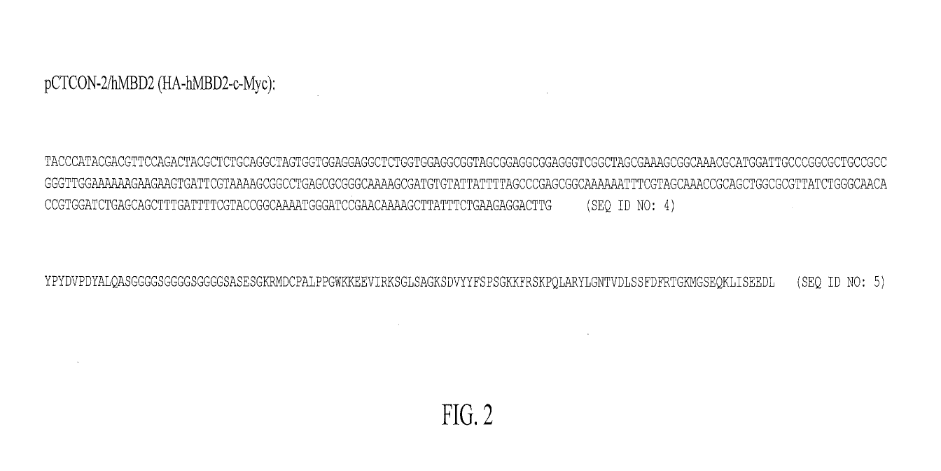

To establish a platform for characterizing and engineering methyl binding domain family proteins, cDNA encoding the MBD domain from hMBD2 was cloned into the pCTCON-2 yeast surface display vector. The construct is expressed as a fusion consisting of Aga2p (for yeast cell surface attachment), HA, MBD, and c-Myc (Chao, Lau, Hackel, Sazinsky, Lippow and Wittrup, 2006) (FIG. 2). Display of hMBD2 was verified by fluorescently labelling the HA and c-Myc epitope tags with ALEXA FLUOR® 647 and 488, respectively, and flow cytometry analysis. The hMBD2 protein was successfully displayed on S. cerevisiae strain EBY100.

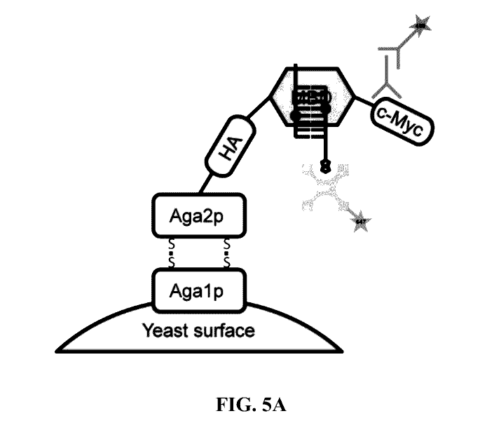

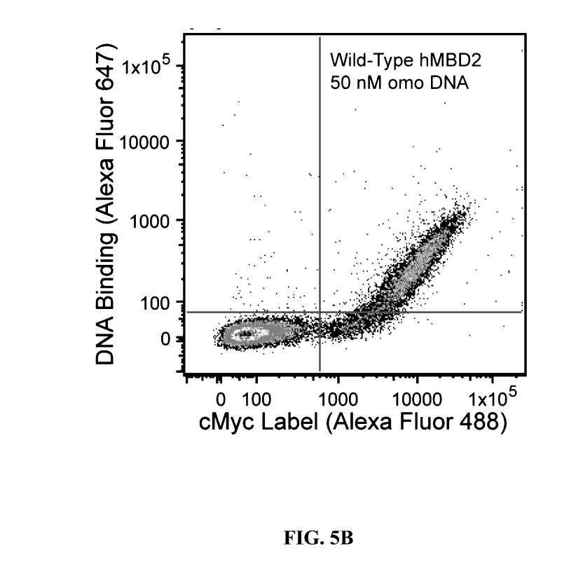

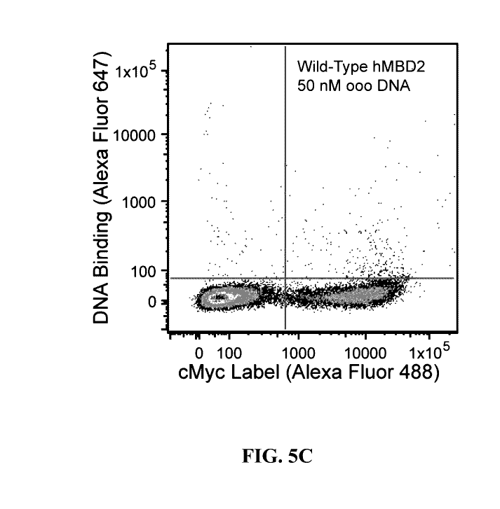

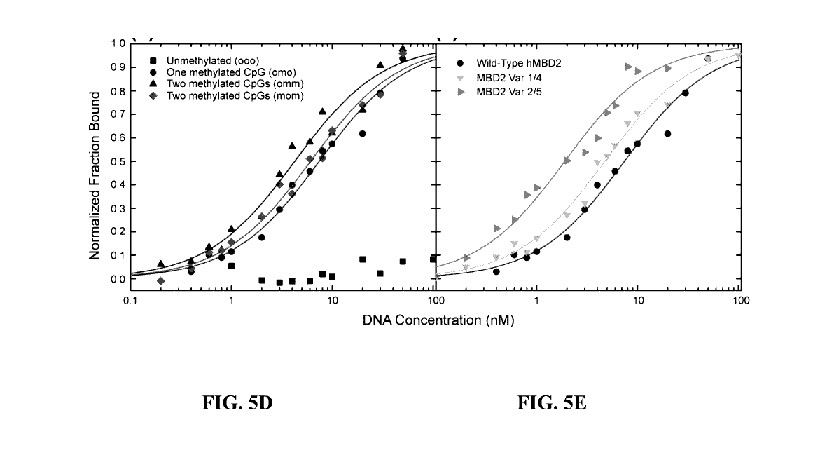

The hMBD2 protein was screened across a range of methylated DNA concentrations to assess relative binding affinities (data not shown). Subsequently, equilibrium binding titration was used to quantitatively determine the affinity and selectivity of the methyl-CpG binding domain of hMBD2. In addition to an anti-c-Myc/ALEXA FLUOR® 488 antibody pair used to show surface display expression, yeast was equilibrated with biotinylated DNA at various concentrations followed by secondary labelling with streptavidin, ALEXA FLUOR® 647 (FIG. 5a).

### Example 2

### Characterizing MBD Binding to DNA Oligonucleotides with Varying Methylation Patterns

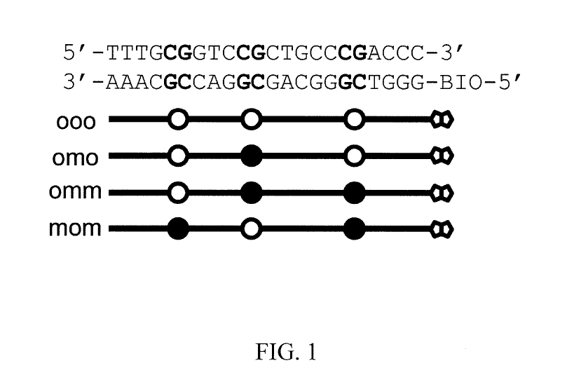

Quantitative equilibrium binding of DNA to yeast displayed hMBD2 proteins was determined using the method described previously (Chao et al., 2006). EBY100 transformed with pCTCON-2/hMBD2 were grown in SDCAA media overnight at 30° C. and 250 rpm. After reaching OD₆₀₀=2-5, cultures were inoculated to OD₆₀₀=1 in SGCAA and incubated at 20° C. and 250 rpm for 40-48 h to induce surface display fusion expression. Induced EBY100 were resuspended to OD₆₀₀=1 in PBSA (1×PBS, 0.1% w/v BSA). Five-hundred thousand EBY100 cells in PBSA were incubated with pre-hybridized DNA (synthesized by Integrated DNA Technologies) at concentrations ranging from 0.06-100 nM in volumes of PBSA ranging from 2225-200 μL to provide a 10-fold molar excess of DNA relative to the number of surface display fusions assuming 5×10⁴ MBD/cell (Chao, Lau, Hackel, Sazinsky, Lippow and Wittrup, 2006). The DNA oligonucleotides used for characterizing the variant hMBD2 polypeptides were derived from the MGMT gene as described previously (Yu, Blair, Gillespie, Jensen, Myszka, Badran, Ghosh and Chagovetz, 2010) and functionalized with biotin on the 5′ end of each target strand to facilitate fluorescence labelling (FIG. 1).

Equilibrium binding was performed at room temperature for 45 min as described previously (Chao, Lau, Hackel, Sazinsky, Lippow and Wittrup, 2006). The binding of methylated DNA to displayed hMBD2 proteins was detected using streptavidin, ALEXA FLUOR® 647 (Life Technologies), and the fraction of EBY100 that expressed the surface display fusions was identified using the chicken anti-cMyc (Gallus Immunotech)/ALEXA FLUOR® 488 goat anti-chicken (Life Technologies) antibody pair. The dissociation constant (K_{d}) for each oligonucleotide was determined from an equilibrium binding titration curve fit obtained after plotting the mean fluorescence of the EBY100 cells displaying MBDs versus each DNA concentration (Chao, Lau, Hackel, Sazinsky, Lippow and Wittrup, 2006). Each reported Kd value is the average of three biological replicates performed on separate days following the same protocol.

The equilibrium dissociation constant for each oligo was determined by fitting the normalized mean fluorescence versus DNA concentration data for each of three biological replicates (FIGS. 5d and 5e). Each thermodynamic binding constant is reported as the average and standard deviation of the fit Kd values from these three independent titrations (Table I). The data for hMBD2 binding to unmethylated (ooo) DNA were unsuitable for fitting because saturation could not be achieved even at micromolar DNA concentrations. These data points have only been included for comparison with concentration-matched methylated oligos (FIG. 5d). Human MBD2's selectivity and weak intrinsic non-specific binding to unmethylated DNA can be seen specifically at one concentration for yeast incubated with 50 nM singly methylated (omo) DNA (FIG. 5b) and matched unmethylated (ooo) DNA (FIG. 5c). Interestingly, hMBD2 binds doubly methylated DNA with a consecutive CpG methylation pattern (omm) with higher affinity (K_{d}=4.2±1.0 nM) than DNA with an alternating meCpG arrangement (mom) (K_{d}=6.5±0.6 nM) (p<0.05). The measured affinities for omo and mom DNA are statistically indistinguishable which implies the kinetics of dissociation occur at a similar rate when the methylated CpG dinucleotides are more distant. These results are consistent with previous observations that MBD2 “prefers more densely methylated DNA (Fraga, Ballestar, Montoya, Taysavang, Wade and Esteller, 2003).”

TABLE 1
Equilibrium dissociation constants for wild-type hMBD2, variant ¼,
and variant ⅖ binding to DNA with one (omo) or two (omm or mom)
methylated CpG sites. Thermodynamic constants were determined
from triplicate equilibrium binding titrations with each MBD variant
displayed on the surface of S. cerevisiae cells.

| | DNA | K_{d} by |
|---|---|---|
| | Methylation | titration |
| MBD Clone | Pattern | (nM) |
| | omo | 5.9 ± 1.3 |
| WT hMBD2 | omm | 4.2 ± 1.0 |
| | mom | 6.5 ± 0.6 |
| Var ¼ | omo | 4.4 ± 0.4 |
| Var ⅖ | omo | 3.1 ± 1.0 |

### Example 3

### Human MBD2 Library Creation Using Error Prone PCR

The GeneMorph II Random Mutagenesis Kit (Agilent) was used to perform epPCR on the hMBD2 gene. To affect 1-3 mutations per MBD2 gene (˜5-15 mutations/kb), 250 ng of target DNA (7.75 μg plasmid construct) was used as the template for the epPCR reaction. The forward 5′-CGA CGA TTG AAG GTA GAT ACC CAT ACG ACG TTC CAG ACT ACG CTC TGC AG-3′ (SEQ ID NO: 19), and reverse 5′-CAG ATC TCG AGC TAT TAC AAG TCC TCT TCA GAA ATA AGC TTT TGT TC-3′ (SEQ ID NO: 20) primer pair (Chao, Lau, Hackel, Sazinsky, Lippow and Wittrup, 2006) was used to produce a 367 bp product. The PCR reaction contained 1×Mutazyme II reaction buffer (Agilent), 40 nmol of each dNTP (New England BioLabs), 125 ng of each primer (Integrated DNA Technologies), 7.75 μg pCTCON-2/hMBD2 construct, and 2.5 U Mutazyme II DNA polymerase (Agilent) in a final volume of 50 μL. The thermocycling profile was as follows: initial denaturation at 95° C. for 2 min followed by 30 cycles of denaturation at 95° C. for 30 sec, annealing at 58° C. for 30 sec, extension at 72° C. for 1 min, and a final extension at 72° C. for 10 min. The epPCR product was gel purified and amplified using standard Taq based PCR to provide sufficient DNA material for library creation via transformation and homologous recombination in EBY100 yeast cells (Chao, Lau, Hackel, Sazinsky, Lippow and Wittrup, 2006).

A random mutant yeast display library of 10⁸ hMBD2-derived clones was created and screened to isolate novel MBD proteins exhibiting increased binding affinity to DNA containing at least one methylated CpG dinucleotide.

### Example 4

### Library Screening for MBD2 Variants with Improved Binding Affinity to Methylated CpGs

The library was screened by incubating a number of EBY100 cells 10-fold greater than the calculated diversity (Chao, Lau, Hackel, Sazinsky, Lippow and Wittrup, 2006). For the first library this corresponded to 2×10⁹ cells for a diversity of 2×10⁸. After the first round of fluorescence activated cell sorting (FACS), the number of cells screened was 10-fold greater than the number collected from the previous sort. Because the starting hMBD2 Kd was less than 10 nM, the library was enriched for high affinity MBD2 variants using a kinetic screen (Boder and Wittrup, 1998). The library was incubated with 100 nM biotinylated omo dsDNA while ensuring a 10-fold molar excess of DNA for 45 min at room temperature in order to saturate surface displayed MBDs with labeled DNA. The cells were then washed, resuspended in PBSA, and incubated with 100 nM unlabeled, competitor omo dsDNA at room temperature to distinguish clones by differences in the degree of labeling due to varying dissociation rate constants and, therefore, binding affinities; concurrently, the cMyc epitope tag of each surface display fusion was labeled with chicken anti-cMyc IgY diluted 1:250. The competition time was determined using the method described previously (Boder and Wittrup, 1998) and increased in successive rounds in the range of 90-120 min. The EBY100 population was washed and labeled using streptavidin, ALEXA FLUOR® 647 and ALEXA FLUOR® 488 goat anti-chicken secondary reagents (both diluted 1:100) on ice for 15 min. The library was washed and resuspended to a density of 10⁷ cells/mL in sterile PBSF for sorting on a MoFlo XDP (Beckman Coulter). Diagonal sort gates were drawn to specify the fraction of the cells collected. This value was decreased from 5%, to 1%, and to 0.1-0.2% over three consecutive rounds of flow cytometry following the method described previously (Boder and Wittrup, 1998). Yeast cells were collected in SDCAA media and subsequently propagated at 30° C. and 250 rpm. A tenfold oversampling of the expanded cells was resuspended in SGCAA media for surface display fusion expression and sorting in the next round of screening. After the third round of FACS, the plasmids encoding the MBD2-derived variants were collected using the ZYMOPREP™ Yeast Plasmid Miniprep II (Zymo Research) and transformed into Mach 1 E. coli cells (Life Technologies). Individual clones were isolated and the MBD2 gene was sequenced using the forward primer 5′-CCC CTC AAC TAG CAA AGG CAG-3′ (SEQ ID NO: 21).

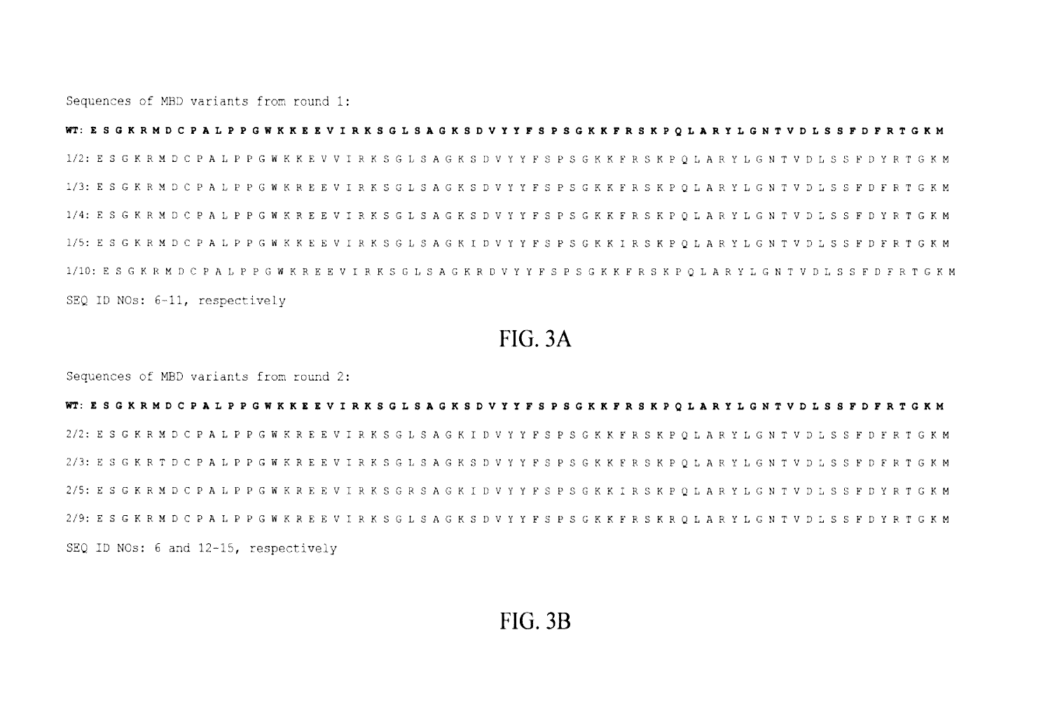

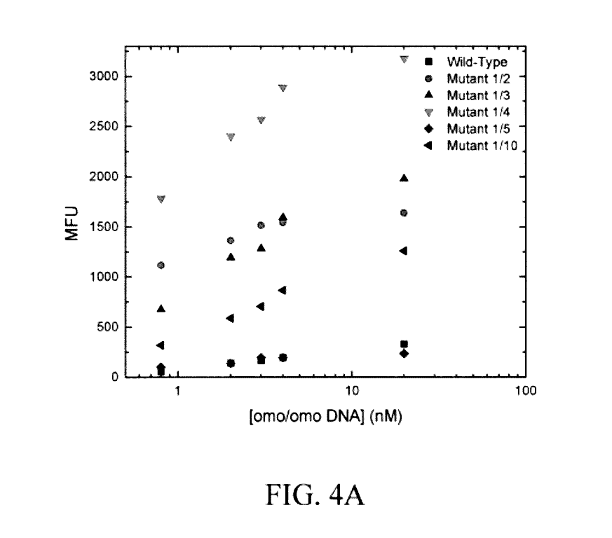

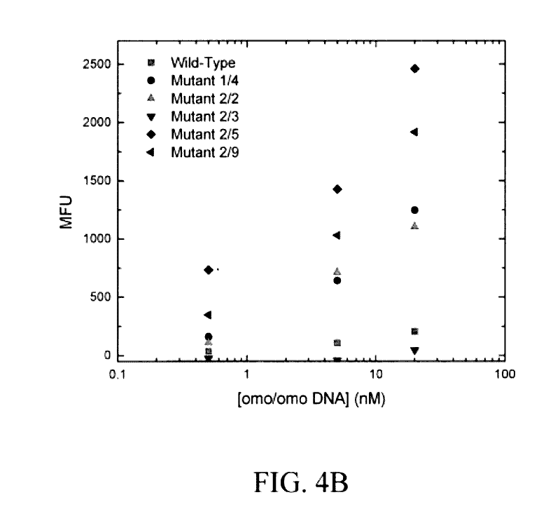

The library was screened by DNA dissociation kinetics such that clones with reduced off rates retained more biotinylated DNA, exhibited greater fluorescence when fluorescently labeled, and were separated using FACS (Boder and Wittrup, 1998). After the first round of epPCR, individual clones were isolated and the gene encoding each MBD variant was sequenced. Six amino acid substitutions (Table II) which combined to produce five unique MBD variants having one or two mutations each (FIG. 3a) were found. One mutation K161R was found in 80% of the clones sequenced. All five variants were screened for binding to singly methylated (omo) DNA in parallel using reduced resolution equilibrium binding titrations and flow cytometry (FIG. 4a).

TABLE 2
Mutations to hMBD2 and the frequency observed during rounds 1 and 2 of MBD
directed evolution by error prone PCR and flow cytometry screening of the
yeast surface display library.

| | Mutation | | | | | | | | |
|---|---|---|---|---|---|---|---|---|---|
| | M150T | K161R | E163V | L170R | S175I | S175R | F187I | P191R | F208Y |
| Round 1 Frequency | — | 0.8 | 0.1 | — | 0.1 | 0.1 | 0.1 | — | 0.2 |
| Round 2 Frequency | 0.11 | 1 | — | 0.11 | 0.22 | — | 0.11 | 0.11 | 0.67 |

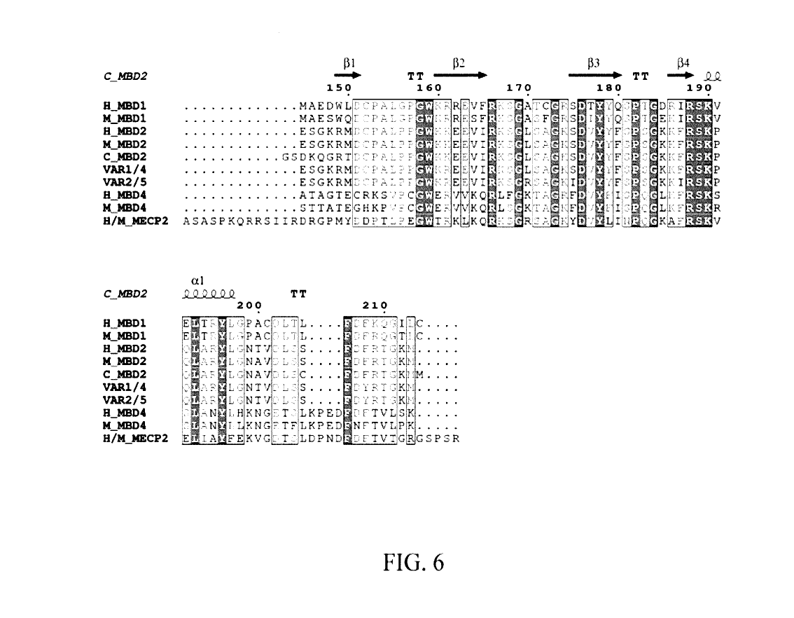

The sequence of the MBD variant ¼, having the highest observed binding affinity, was aligned with the wild-type primary structure (FIG. 6) and performed complete equilibrium binding titrations to omo DNA in triplicate to quantitatively determine K_{d} (FIG. 5e) (Table I). Binding affinity improved approximately 25% from wild-type hMBD2 to 4.4±0.4 nM although not statistically different.

After screening the first library, the plasmids collected from the final sort were subjected to a second round of mutagenesis by epPCR as described above to create another library with a calculated diversity of 1×10⁸. This second library was screened using the same protocol above for the purpose of finding additional mutations giving rise to higher affinity MBD proteins.

Three new amino acid substitutions were observed following this round of evolution (Table II) as well as new combinations of mutations observed previously. The K161R mutation was present in every variant sequenced, and the F208Y was found in 67% of variants up from a 20% frequency in the first round. The four new MBD variants had two to five mutations each (FIG. 3b). The highest affinity MBD variant was determined using the rapid flow cytometry screen described above (FIG. 4b); MBD variant 2/5 contains five mutations (FIG. 6) and has an affinity (K_{d}=3.1±1.0 nM) approximately two-fold greater than wild-type hMBD2 (FIG. 5e). Given the combination of mutations, this variant may have potentially arisen from recombination of variants 1/4 and 1/5 from the first round of evolution.

### Example 5

### Bacterial Expression of MBD2 Variant Proteins

The cDNA for MBD2 variant 2/5 was codon optimized for expression in E. coli (Gene Art-Life Technologies) and used to create an MBD-GFP fusion analogous to that reported previously (Yu, Blair, Gillespie, Jensen, Myszka, Badran, Ghosh and Chagovetz, 2010). The protein consists of an N-terminal His₆-tag followed by the nuclear localization sequence PKKKRKV, the MBD2 variant 2/5, a hemagglutinin (HA) tag, and a C-terminal enhanced green fluorescent protein (GFP) tag. A BsaI restriction site was included immediately preceding the MBD2 variant 2/5 to facilitate concatenation. The cDNA encoding the fusion was synthesized as a gBlock with flanking 5′ EcoRI and 3′ XhoI restriction sites plus four nucleotide overhangs, double digested, ligated into the pET-30b+ vector, and transformed into Mach 1 E. coli cells (Life Technologies). The miniprepped plasmid was subsequently transformed into BL21 (DE3) Tuner E. coli cells (Novagen) for expression.

To create the MBD2 variant 2/5 multimer, a second gBlock consisting of the codon optimized cDNA for the MBD followed by the cDNA for a (Gly₄-Ser)₂ linker with flanking 5′ and 3′ BsaI restriction sites plus six nucleotide overhangs on each end was designed. Both the pET-30b+/MBD2 variant 2/5 plasmid and second gBlock were digested with BsaI (New England Biolabs) and ligated using T4 DNA ligase (New England Biolabs) such that the digested gBlock was in large molar excess. The ligation product was transformed into Mach 1 E. coli cells and plated onto LB agar plates supplemented with kanamycin. Individual clones were screened for the number of incorporated MBD variant 2/5 monomer units on the basis of the size of the fragment obtained following double digestion with EcoRI and XhoI. The plasmid encoding the 3×MBD2 variant 2/5-GFP protein was transformed into BL21 (DE3) Tuner E. coli cells (Novagen) for expression. The 1× and 3×MBD2 variant 2/5 proteins were expressed (Boyd et al., 2012) and purified under denaturing conditions with on-column refolding (Jorgensen, Adie, Chaubert and Bird, 2006) using the protocols described previously.

### Example 6

### MBD Surface Binding Experiments and Affinity Determination

Clear glass slides coated with an agarose film were prepared (Afanassiev et al., 2000) and printed (Heimer, Shatova, Lee, Kaastrup and Sikes, 2014) with pre-hybridized ooo probe/ooo target, omo probe/omo target, and omm probe/omm target oligonucleotides at 10 μM concentration in 3×SSC as described previously. A circular, 9 mm diameter isolator well was cut from Scotch 3M 665 tape and affixed to the biochip to define each test area. Each biochip was then rinsed under a stream of DI water and blown dry using compressed nitrogen gas. Biochips ready for testing were stored in the vacuum desiccator until needed.

N×MBD proteins were diluted in binding buffer (20 mM HEPES, pH 7.9, 3 mM MgCl₂, 10% v/v glycerol, 1 mM dithiothreitol, 100 mM KCl, 0.1% w/v BSA, 0.01% Tween-20, and 1 μM ssDNA) and pre-incubated for 10 min at room temperature. Each 40 μL N×MBD dilution was added to a separate test area and incubated for 40-45 min in a humid chamber at ambient temperature (approximately 20-22° C.). Each slide was washed sequentially with 1×PBS/0.1% v/v Tween 20 (PBST), 1×PBS, and 18 M. DI water and blown dry using compressed nitrogen gas. The monoclonal mouse HA.11 clone 16B12 antibody (BioLegend) was diluted 1:100 in 1×PBS/0.1% w/v BSA (PBSA), added to each test area, and incubated for 10 min at 4° C. in a humid chamber pre equilibrated to temperature. The slide was washed and dried as described previously. The secondary ALEXA FLUOR® 647 goat, anti-mouse antibody was diluted 1:100 in PBSA, added to each test area, and incubated for 10 min at 4° C. in a humid chamber pre equilibrated to temperature. The slide was washed and dried as described previously before scanning with a GenePix 4000B fluorescent microarray scanner (Molecular Devices). Each fluorescence image was analyzed using ImageJ (NIH). The mean fluorescence intensity for each spot was determined by adjusting the threshold of the image to include the entire spot area and averaging the constituent pixel intensities. The values for all spots of the same DNA methylation pattern were averaged and plotted versus the N×MBD concentration in order to fit the data and determine the apparent equilibrium dissociation constant K_{d,app}.

### Example 7

### Structural Modelling for MBD Variants with Improved Binding Affinity to meDNA

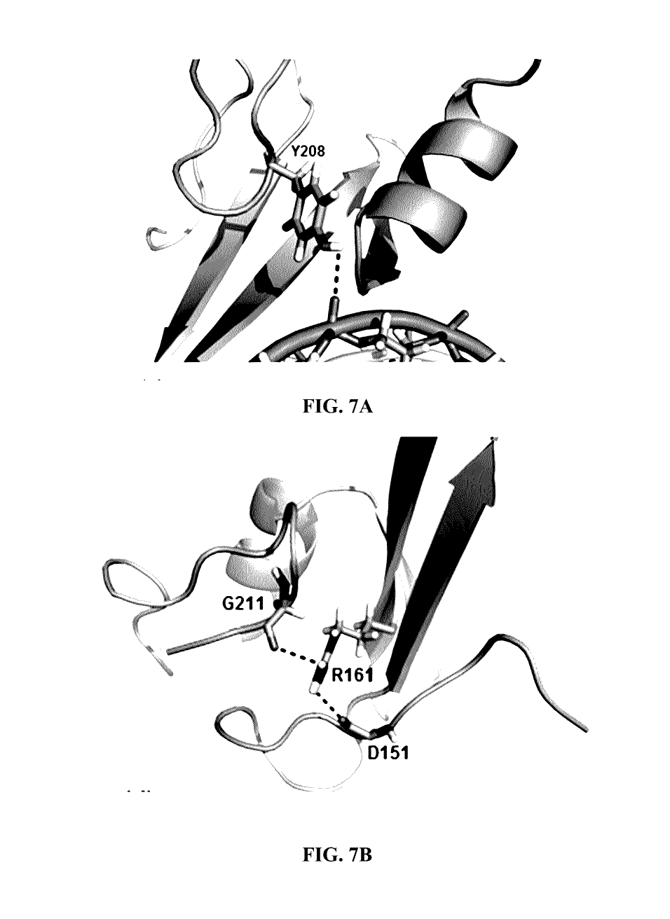

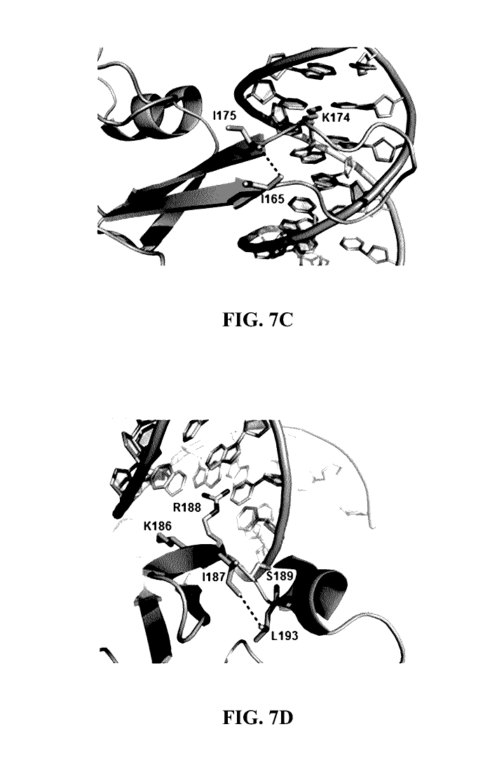

In order to determine the molecular basis of the observed affinity improvements, the SWISS-MODEL system (Biasini et al., 2014) and the published chicken MBD2 NMR structure (2KY8 PDB) (Scarsdale, Webb, Ginder and Williams, 2011) was used to generate a homology model of the MBD2 variant 2/5. The kinetic library screening method is used to isolate variants with decreased off-rates (Boder and Wittrup, 1998). As such, forming new, non-covalent protein-DNA interactions slows the rate of MBD-DNA dissociation and results in improved binding affinity. In the case of hMBD2, mutation of phenylalanine to tyrosine at the 208 position adds a para substituted hydroxyl group to the aromatic side chain which donates a hydrogen bond to the DNA phosphate backbone (FIG. 7a). Similarly, the L 170R mutation restores an ionic interaction between the positively charged guanidinium group and the phosphate backbone of DNA native to MeCP2 but not present in wild-type MBD2 variants (Ohki et al., 2001, Scarsdale, Webb, Ginder and Williams, 2011).

The frequency of which the K161R mutation is observed, if used as a surrogate for fitness, may indicate it is the most significant residue of those found affecting MBD binding affinity. Despite being the highest affinity wild-type MBD reported (Fraga, Ballestar, Montoya, Taysavang, Wade and Esteller, 2003), MBD2 is the only wild-type human or mouse MBD having a lysine at this position instead of an arginine. The hMBD2 K161 side chain forms a single hydrogen bond between its e-amino group and the backbone of G211 in the wild-type protein (Scarsdale, Webb, Ginder and Williams, 2011). Mutating this residue to arginine substitutes a resonance stabilized guanidium group for the e-amino which allows for the formation of a second hydrogen bond to the backbone of D151 (FIG. 7b). Together these two interactions allow R161 to stabilize the N- and C-terminal ends of the protein at the interface with the 3 sheet (see secondary structure in FIG. 6). Missense mutation of the homologous residue R106 to tryptophan in MeCP2 has been implicated in the development of Rhett syndrome (Ho et al., 2008). Further, the R106W mutation has been shown to thermally destabilize the motif and reduce the binding affinity to methylated DNA by inducing changes in the MBD secondary structure (Ghosh et al., 2008).

The two mutations to isoleucine S1751 and F187I appear to exist within a similar context in the MBD structure. Both are adjacent to residues known to form base-specific interactions: K174 with the guanine downstream of the CpG and R188 directly with the methylated CpG, respectively (Scarsdale, Webb, Ginder and Williams, 2011). D176 was also shown to form a CH . . . O hydrogen bond to the methyl group of 5 mC in homologous h/mMeCP2 over a similar distance (˜3.5 Å) (Ho, McNae, Schmiedeberg, Klose, Bird and Walkinshaw, 2008). Further, I187 is one member of the four amino acid sequence KIRS in which all three other residues interact with the bound DNA strand. In both instances, the hydrophobic isoleucine side chains are oriented nearly opposite of those interacting with the DNA. The I175 side chain appears to engage in a hydrophobic interaction with I165 at the C-terminal end of the second β-strand (FIG. 7c). Likewise, I187 forms a similar hydrophobic interaction with L193 residing in the a-helix (FIG. 7d). The mechanism for the affinity enhancement from these mutations is unclear; however, it may be due to further stabilization of the local MBD structure or that the hydrophobic side chain positioning opposite the DNA-interacting side chains may allow the DNA binding residues greater freedom to form interactions with bound DNA.

### Example 8

### Affinity Enhancement by Concatenation for Interfacial Binding Applications

Starting with a wild-type mMBD1 (K_{d}=30 μM), others have reported a 60-fold improvement in MBD affinity (K_{d}=0.5 μM) for singly methylated DNA by concatenating four mMBD1s into a single peptide (Jorgensen, Adie, Chaubert and Bird, 2006). Adopting this established method, the highest affinity monomeric MBD variant (MBD 2/5, FIG. 3b) was concatenated in order to increase its probability of forming MBD-meDNA interactions as well as enabling it to form multiple interactions with DNA strands having multiple sites of CpG methylation. Each MBD 2/5 multimer was expressed as an enhanced green fluorescent protein (GFP) fusion to facilitate fluorescence detection, enhanced soluble expression in E. coli, and quantification of purified protein yield by 488 nm absorbance measurement (Boyd, Heimer and Sikes, 2012, Yu, Blair, Gillespie, Jensen, Myszka, Badran, Ghosh and Chagovetz, 2010).

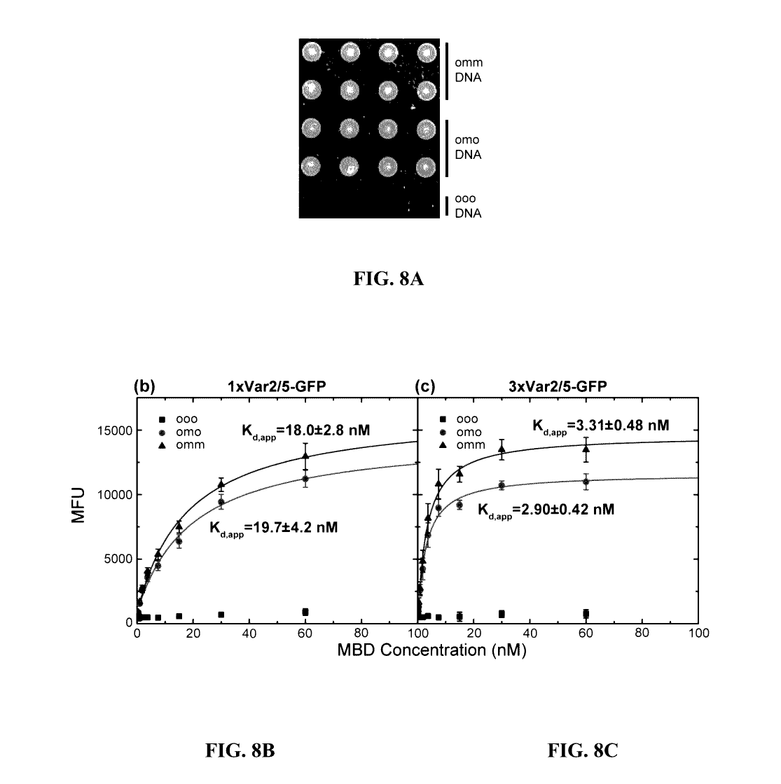

In order to further the development of high-performance, interfacial epigenotyping assays (Heimer, Shatova, Lee, Kaastrup and Sikes, 2014, Yu, Blair, Gillespie, Jensen, Myszka, Badran, Ghosh and Chagovetz, 2010), N×MBD (i.e., multimeric) variants were evaluated on agarose coated slides (Afanassiev, Hanemann and Wölfl, 2000) with immobilized dsDNA having no (ooo), one (omo), or two (omm) methylated CpG dinucleotides. The bound MBDs were labeled with an anti-HA/ALEXA FLUOR® 647 antibody pair and scanned them (FIG. 8a). The apparent, equilibrium dissociation constant (K_{d,app}) was determined for each N×MBD by plotting the mean fluorescence from each group of spots versus the MBD concentration applied to the test site and fitting a monovalent, equilibrium binding model to the data. The 1× variant was found to bind singly methylated DNA with K_{d,app}=19.7±4.2 nM and doubly methylated DNA with K_{d,app}=18.0±2.8 nM (FIG. 8b). Both these values are within each other's error and only show a small improvement in 1×MBD binding to doubly methylated DNA. The 3×MBD variant exhibits an approximately 6-fold improvement in binding to singly methylated DNA with K_{d,app}2.90±0.42 nM and doubly methylated DNA with K_{d,app}=3.31±0.48 nM while exhibiting negligible binding to unmethylated DNA (FIG. 8c).

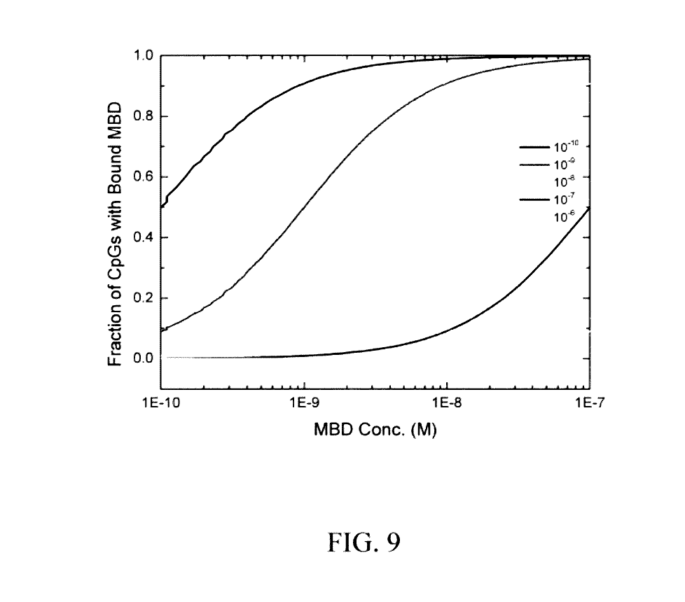

Such binding affinity improvements while maintaining specificity allows us to preserve solution-like binding characteristics in a useful interfacial format where surface effects as well as MBD loss during wash steps can reduce the fractional MBD coverage. The fractional coverage of single methylated CpGs as a function of concentration for MBD proteins with varying Kd was estimated using a Langmuir adsorption model (FIG. 9). The MBD proteins described here with single-digit nanomolar dissociation constants (10⁻⁹ M) can provide fractional coverages several fold higher than other MBDs having Kd values on the order of 100 nM (10⁻⁷ M) (Cipriany, Zhao, Murphy, Levy, Tan, Craighead and Soloway, 2010, Jørgensen, Adie, Chaubert and Bird, 2006, Yu, Blair, Gillespie, Jensen, Myszka, Badran, Ghosh and Chagovetz, 2010).

### Example 9

### Recognition and Binding of Hemi-Methylated DNA

Developing a protein that will recognize hemi-methylated DNA, where the cytosine bases are only methylated on one of the two DNA strands, would allow the detection of a methylated sequence from a patient's sample bound to an unmethylated capture probe. A library created from human MBD2 as described above was used as a starting point. Variants with improved affinity for hemi-methylated DNA were isolated and analyzed in a yeast surface display construct.

Characterization of Binding Affinities Using Yeast Surface Display MBD proteins were displayed on the surface of EBY100 S. cerevisiae yeast cells as described above. The cells containing the pCTCON-2 vector with the MBD insert were grown overnight in SDCAA medium at 30° C. and 250 rpm. To induce protein expression, after the SDCAA cultures reached an OD600 between 2 and 5, the cells were resuspended in SGCAA medium to an OD600 of 1 and incubated at 20° C. and 250 rpm for 36-48 hours. The cells were then resuspended in PBS with 0.1% BSA and an equilibrium binding titration was performed by incubating the cells expressing the MBD protein with biotinylated DNA oligomers at a range of concentrations between 0.05 and 100 nM for 45 min at room temperature. Total reaction volumes were chosen to ensure 10-fold excess of DNA in each sample, calculated based on the protein expression level identified by Chao et al. (Chao et al., 2006). Expressed protein and bound DNA were labeled with chicken anti-cMyc/AlexaFluor-488 and streptavidin-AlexaFluor-647, respectively. The extent of binding was evaluated using flow cytometry, and dissociation constants were calculated using the method described by Chao et al. (Chao et al., 2006) Screening the MBD Library for Improved Affinity for Hemi-Methylated DNA To enrich for protein variants that bind to hemi-methylated DNA, biotinylated DNA with a single methylated cytosine on one strand was incubated with streptavidin conjugated magnetic beads. The DNA concentration in the 1 ml reaction was 55 nM. A total of 4×10⁹ cells expressing the MBD library were incubated with the DNA covered beads for 2 hours at 4° C. to capture those expressing proteins with good binding characteristics. After the incubation, the beads with cells attached were separated from unbound cells with a magnet and resuspended in SDCAA medium, pH 4.5, supplemented with pen-strep (1:100 dilution). The captured cells were grown overnight at 30° C. and 250 rpm. The bead selection was repeated with 2×10s cells from the enriched library. After the second selection with magnetic beads, the cells were again grown up and protein expression was induced. Two additional selections for hemi-methylated DNA were performed using fluorescence-activated cell sorting (FACS). For the first FACS selection, binding reactions were prepared as described for characterization by flow cytometry and a gate was drawn during sorting to capture the top 1% of cells. This top 1% was defined using a diagonal sort window, as described by Chao et al. (Chao et al., 2006). In the second FACS selection, the top 0.37% of the cells were isolated. The plasmids encoding the selected proteins were extracted using the ZYMOPREP™ Yeast Plasmid Miniprep II kit, transformed into Mach 1 E. coli, and grown on LB plates containing 100 μg/ml ampicillin. Ten single colonies were selected, and for each of these colonies, the MBD insert was sequenced. After sequencing, plasmids containing unique clones were transformed into EBY100 S. cerevisiae and expressed on the surface. To compare the clones, binding reactions were performed as described above with two DNA concentrations, 10 nM and 50 nM. After a comparison of binding affinities among the isolated clones, titrations were performed to determine the dissociation constant of the top performing variant.

### Soluble Protein Expression

The sequence encoding the top performing variant, h4 (see Table 3 below), was PCR amplified from the pCTCON-2 vector using Phusion HF polymerase with the forward primer 5′-GCCTGAATTCTGAAAGCGGCAAACG-3′, which includes an EcoRI restriction site, and the reverse primer 5′-CATTTTGCCGGTACGATAATCAAAGCTGCTC-3′. In this reaction, the DNA was denatured at 95° C. for 6 min, then 30 cycles were performed with 30 sec each of denaturation at 95° C., annealing at 56° C., and extension at 72° C. A 10 min final extension was performed at 72° C. Splicing by overlap extension was used to append an eGFP tag and a biotin accepter sequence to MBD2 variant h4. First, a 3-primer PCR reaction was used to add a linker sequence to the MBD variant. This reaction used the forward primer 5′-GCCTGAATTCTGAAAGCGGCAAACG-3′, the long reverse primer 5′-CGTAGTCTGGCACGTCGTATGGGTACATTTTGCCGGTACGATAATCAAAGCTG-3′ for adding the linker group, and the short reverse primer 5′-CGTAGTCTGGCACGTCGTATGGG-3′ for amplifying the product containing the linker group with the same PCR conditions as the first reaction. The eGFP tag and biotin accepter sequence were amplified from another plasmid using the forward primer 5′-TACCCATACGACGTGCCA-3′and the reverse primer 5′-TGGTGCTCGAGTTTATTCATGC-3′, which added an XhoI restriction site. The eGFP reaction proceeded as described above except the annealing temperature was reduced to 52° C. and the extension time increased to 1 min. For the splicing by overlap extension reaction, the forward primer 5′-GCCTGAATTCTGAAAGCGGCAAACG-3′ and reverse primer 5′-TGGTGCTCGAGTTTATTCATGC-3 were used to amplify the full MBD-GFP fusion protein using touchdown PCR. An annealing temperature of 61° C. was used for the first cycle, and this temperature was decreased by 1° C. for each of the next eight cycles. The final annealing temperature of 53° C. was then used for an additional 30 cycles. The resulting PCR product was cloned into the pET30b vector using the EcoRI and XhoI restriction sites. The pET30b vector containing the insert was transformed into DE3 Tuner E. coli and grown in LB broth supplemented with Kanamycin. To express the fusion protein, the cells were grown in TB medium to an OD600 of 0.6 and then protein expression was induced by the addition of 0.05 mM IPTG. The cells were incubated at 20° C. for 16 hours, pelleted, and lysed using BugBuster HT protein extraction reagent according to the manufacturer's protocol for soluble protein.

### Biochip Experiments

Glass slides were coated with 0.2% SEAKEM® LE agarose (Lonza) and arrays of pre-hybridized DNA were printed, as described by Heimer et al. (Heimer et al., 2014). Each slide contained rows with ooo probe/omm target, omo probe/ooo target, and ooo probe/ooo target DNA. The slides were left to dry in a vacuum desiccator overnight. Wells were cut from Scotch 3M tape and placed around the arrays on the slide. The wells were rinsed with 18 MΩ DI water and dried under compressed air. Blocking was performed by incubating the wells with 40 μl of 1% BSA at room temperature for 15 min. After the blocking reaction, the wells were rinsed with PBS and 18 MΩ DI water and dried with compressed air before 40 ul of the clarified cell lysate containing MBD2 variant h4, diluted in binding buffer (20 mM HEPES, pH 7.9, 3 mM MgCl2, 10% (v/v) glycerol, 1 mM dithiothreitol, 100 mM KCl, 0.1% (w/v) BSA, 0.01% Tween-20), was added. The DNA arrays and protein solution were incubated at room temperature for 45 minutes, after which the wells were washed consecutively with PBS/0.1% Tween 20, PBS, and 18 M DI water and dried with compressed air. Bound protein was labeled with streptavidin-ALEXA-FLUOR® 647 diluted 1:100 in PBSA for 10 min at 4° C. and the wells were washed and dried again, as described above. All incubation steps were performed in a humid chamber that had been equilibrated to the desired incubation temperature. Fluorescence was detected using a GenePix 4000B scanner (Molecular Devices) with 635 nm excitation. Quantitative results were obtained by calculating the mean fluorescence and background fluorescence for each spot within the DNA array using the GenePix 6.1 software. For each methylation pattern, the fluorescence intensity was averaged over all of the spots within the well.

### Characterization of Binding Affinity of Wild-Type MBD2 for Hemi-Methylated DNA

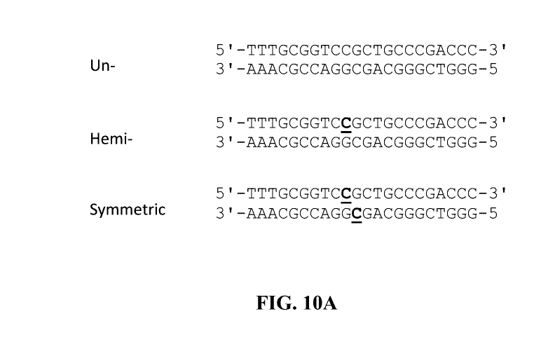

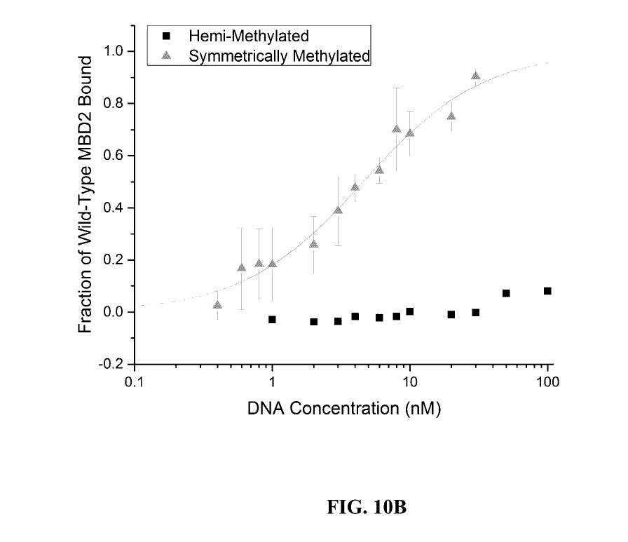

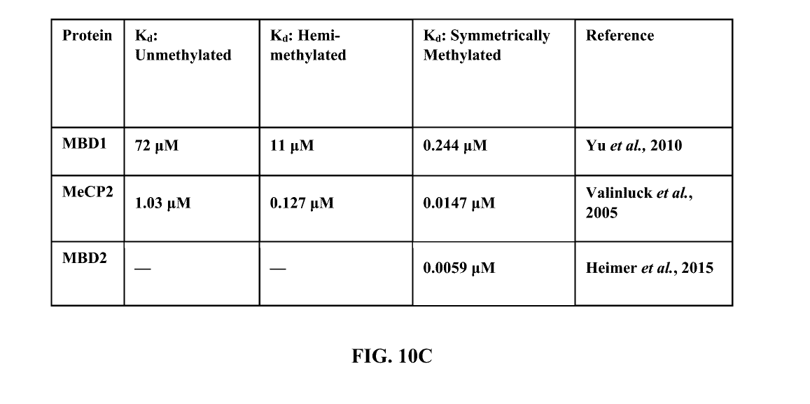

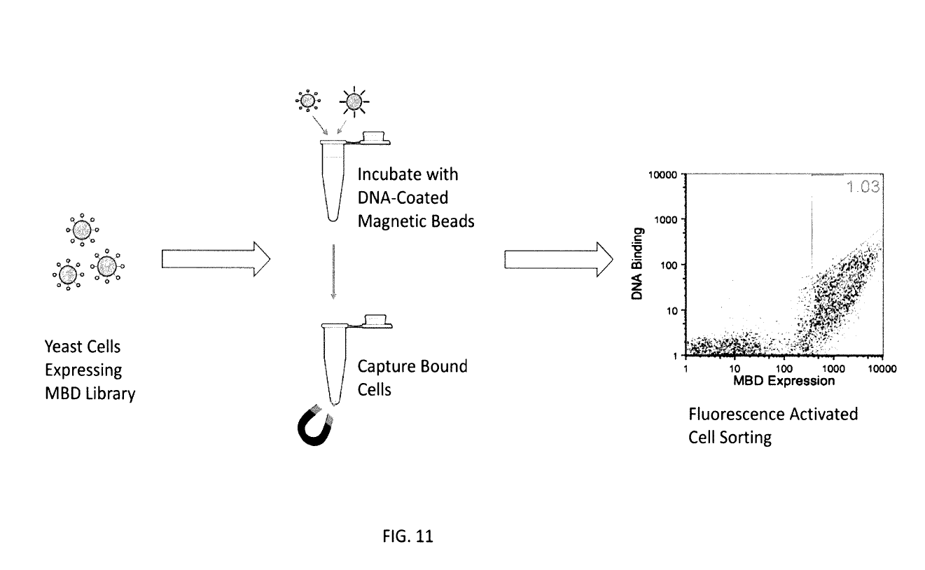

To characterize the binding affinity, the MBD proteins were displayed on the surface of S. cerevisiae using the pCTCON-2 vector. The binding affinity of wild-type human MBD2 toward a DNA oligo with a single methyl group on one strand was evaluated using equilibrium binding titrations with flow cytometry. The sequence and methylation patterns of the test DNA used for characterization are shown in FIG. 10a. These sequences are based on a region of the MGMT gene. In the equilibrium binding titrations, expression of the MBD protein was verified by labeling the cMyc tag of the fusion protein with ALEXAFLUOR® 488, and the cells expressing the protein were incubated with the biotinylated DNA at a range of concentrations and labeled with streptavidin, ALEXAFLUOR® 647. As shown in FIG. 10b, the wild type MBD2 protein binds to symmetrically methylated DNA with high affinity but shows almost no binding to the hemi-methylated DNA sample, even at concentrations as high as 100 nM. Because binding was not observed, a dissociation constant could not be determined using this method. The results demonstrating that MBD2 binds to symmetrically methylated DNA with much higher affinity than hemi-methylated DNA agree with previously reported data for MBD1 and MeCP2, shown in FIG. 10c, that show affinity differences of an order of magnitude or more between the two methylation states.

### Affinity Maturation and Screening

Beginning with the error-prone PCR library generated as described above, variants of the protein human MBD2 were displayed on the surface of yeast cells, and those with improved affinity for hemi-methylated DNA were selected using an equilibrium binding assay. The selection process is depicted in FIG. 11. In the early rounds of selection, cells expressing the MBD library were incubated with hemi-methylated DNA attached to magnetic beads, allowing the cells that bind to the DNA to be separated from the larger library. In later rounds, the cells isolated from the magnetic bead selections were incubated with biotinylated, hemi-methylated DNA that was then labeled with a streptavidin-conjugated fluorophore. In this assay, cells expressing proteins with the highest affinities had the largest number of fluorophore-labeled DNA molecules attached, giving the brightest signal during flow cytometry. These cells were isolated using fluorescence-activated cell sorting (FACS).

The amino acid sequences of the proteins isolated after the selection procedure are shown in Table 3. All of the variants isolated had the K161R mutation and 70% had the F208Y mutation, two mutations that, without wishing to be bound by any particular theory, allow for the formation of an additional hydrogen bond to stabilize the protein structure and to bind to the DNA backbone, respectively. The F187I mutation, which is adjacent to the arginine residue that interacts with the methylated cytosine base, was also found in 50% of the isolated proteins.

TABLE 3
Sequences of MBD Variants Isolated (with mutations
shown in underline)

| | |
|---|---|
| WT | ESGKRMDCPALPPGWKKEEVIRKSGLSAGKSDVYYFSPSGKKFRSK |
| | LPQARYLGNTVDLSSFDFRTGKM (SEQ ID NO: 6) |
| h1 | ESGKRMDCPALPPGWKREEVIRKSGLSAGKIDVYYFSPSGKKIRS |
| | KPQLARYLGNTVDLSSFDFRTCKM (SEQ ID NO: 22) |
| h2 | ESGKRMDCPALPPGWKREEVIRKSGLSAGKSDVYYFSPSGKKFRSK |
| | PQLARYLGNTVDLSSFDYRTGKM (SEQ ID NO: 9) |
| h3 | ESGKRMDCPALPPGWKREEVIRKSGLSAGKSDVYYFSPSGKKFRSK |
| | PQLARYLGNTVDLSSFDFRTGKM (SEQ ID NO: 8) |
| h4 | ESGKRMDCPALPPGWKREEVIRKSGLSAGKSDVYYFSPSGKKIRSK |
| | PQLARYLGNSVDLSSFDYRTGKM (SEQ ID NO: 23) |
| h5 | ESGKRMDCPALPPGWKREEVIRKSGLSAGKSDVYYYSPSGKKFRSK |
| | PQLARYLGNTVDLSSFDYRTGKM (SEQ ID NO: 24) |
| h6 | ESGKRMDCPALPPGWKREEVIRKSGLSAGKIDVYYFSPSGKKFRS |
| | KPQLARYLGNTVDLSSFDFRTGKM (SEQ ID NO: 12) |
| h7 | ESGKRMDCPALPPGWKREEVIRKSGLSAGKSDVYYFSPSGKKIRS |
| | KPQLARYLGNTVDLSSFDYRTGKM (SEQ ID NO: 25) |
| h8 | ESGKRMDCPALPPGWKREEVIRKSGLSAGKIDVYYFSPSGKKIRS |
| | KPQLARYLGNTVDLSSFDYRTGKM (SEQ ID NO: 26) |

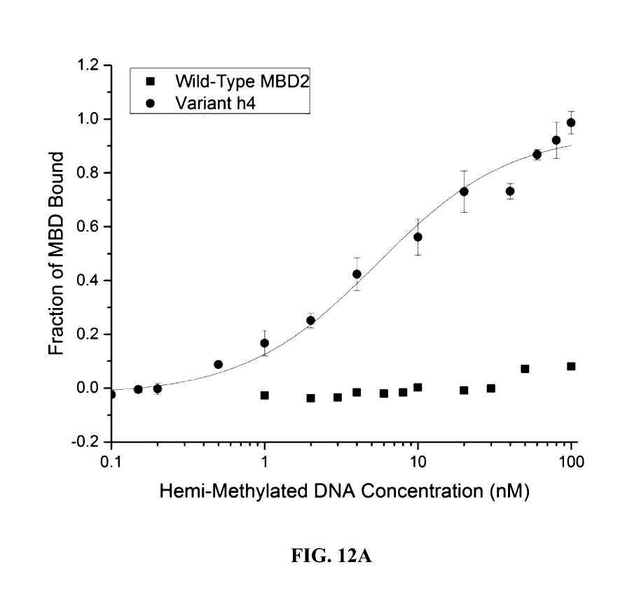

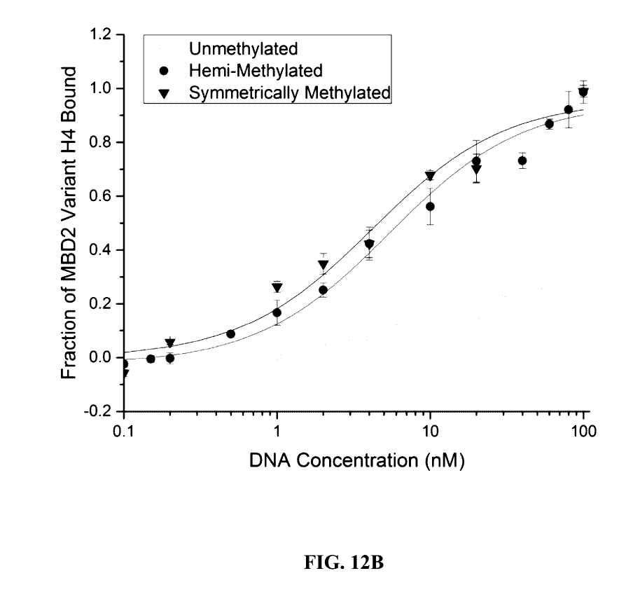

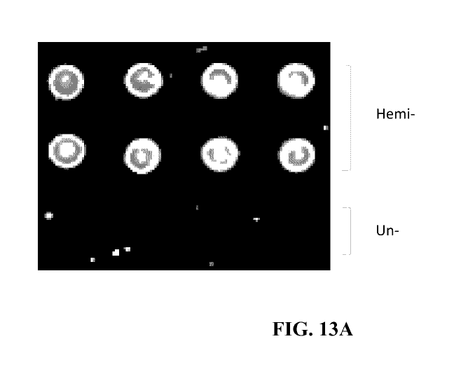

Unique protein variants were compared (data not shown), and the top-performing protein, variant h4, was characterized by equilibrium binding titrations. FIG. 12a shows the improvement in the binding affinity of the engineered protein for hemi-methylated DNA over that of the wild-type MBD2 protein. Completion of equilibrium binding titrations in triplicate gives a dissociation constant of 5.6±1.4 nM, a value nearly identical to the wild type protein's dissociation constant with symmetrically methylated DNA. FIG. 12b shows that the new protein binds to hemi-methylated DNA and symmetrically methylated DNA with similar affinity while retaining good specificity for these constructs over unmethylated DNA.

The fourth mutation, T200S, in variant h4, is a small change from a threonine to the slightly smaller serine and is located far from the DNA binding site. This residue is not conserved across the MBD family: it is found as alanine in MBD1, threonine in human MBD2, asparagine in MBD4, and valine in MeCP2. However, none of the wild type MBD proteins nor any of the proteins isolated from the library except for variant h4 have the S200 residue. Nevertheless, this mutation appears to play an important role in binding to hemi-methylated DNA.

### Biochip Assay

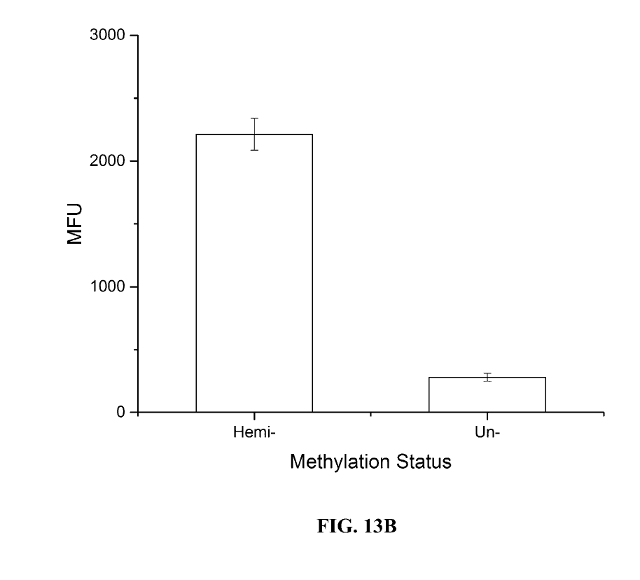

To determine whether the new protein can function to distinguish between hemi-methylated and unmethylated DNA in the interfacial binding assays, binding experiments were performed with soluble MBD2 variant h4 and DNA arrays printed on agarose-coated glass slides. The MBD2 variant h4 was cloned into the pET30b bacterial expression vector and expressed as a fusion protein with eGFP and a biotin acceptor sequence. The slides were printed with hemi-methylated DNA as well as unmethylated DNA. Biotinylated MBD bound to the DNA was labeled with streptavidin, ALEXAFLUOR® 647 and detected by fluorescence imaging. In the resulting image, found in FIG. 13a, MBD bound to the hemi-methylated DNA is easily visible while the spots printed with unmethylated DNA show little binding and are very difficult to identify by eye, a visual distinction that is confirmed by the quantitative results shown in FIG. 13b. Specificity over unmethylated DNA was retained with the variants of the present invention, and the variants were shown to distinguish between hemi-methylated and unmethylated DNA in an interfacial binding assay.

These results demonstrate that variants of the present invention can be used in place of the wild-type MBD proteins used in previously developed epigenotyping assays and that unmethylated DNA probes can now be used instead of methylated probes in these assays. Because methylated DNA probes must be specially synthesized and are much more costly than unmethylated probes, an assay that doesn't require them can be developed more quickly and easily into a method suitable for clinical use. Such binding assays could be extremely valuable as an alternative to the chemical conversion-based methods currently used for clinical methylation analyses that have many disadvantages, such as DNA degradation during sample treatment.

While this invention has been particularly shown and described with references to preferred embodiments thereof, it will be understood by those skilled in the art that various changes in form and details may be made therein without departing from the scope of the invention encompassed by the appended claims.

### REFERENCES

Afanassiev V., Hanemann V. and Wölfl S. (2000) Preparation of DNA and protein micro arrays on glass slides coated with an agarose film. Nucleic Acids Research, 28, e66. First published on, doi: 10.1093/nar/28.12.e66.
Biasini M., Bienert S., Waterhouse A., Arnold K., Studer G., Schmidt T., Kiefer F., Cassarino T. G., Bertoni M., Bordoli L. et al. (2014) SWISS-MODEL: modelling protein tertiary and quaternary structure using evolutionary information. Nucleic Acids Research. First published on, doi: 10.1093/nar/gku340.
Boder E. T. and Wittrup K. D. (1998) Optimal Screening of Surface-Displayed Polypeptide Libraries. Biotechnology Progress, 14, 55-62. First published on, doi: 10.1021/bp970144q.
Boyd M. E., Heimer B. W. and Sikes H. D. (2012) Functional heterologous expression and purification of a mammalian methyl-CpG binding domain in suitable yield for DNA methylation profiling assays. Protein Expression and Purification, 82, 332-338. First published on, doi: 10.1016j.pep.2012.01.016.
Brinkman A B, Simmer F, Ma K, Kaan A, Zhu J, Stunnenberg H G. Whole-genome DNA methylation profiling using MethylCap-seq. Methods. 2010; 52(3):232-6. doi: 10.1016/j.ymeth.2010.06.012.
Chao G., Lau W. L., Hackel B. J., Sazinsky S. L., Lippow S. M. and Wittrup K. D. (2006) Isolating and engineering human antibodies using yeast surface display. Nat Protocols, 1, 755-768. First published on, doi: http://www.nature.com/nprot/journal/vl/n2/suppinfo/nprot.2006.94_S1.html.
Cipriany B. R., Murphy P. J., Hagarman J. A., Cerf A., Latulippe D., Levy S. L., Benitez J. J., Tan C. P., Topolancik J., Soloway P. D. et al. (2012) Real-time analysis and selection of methylated DNA by fluorescence-activated single molecule sorting in a nanofluidic channel. Proceedings of the National Academy of Sciences, 109, 8477-8482. First published on, doi: 10.1073/pnas. 1117549109.
Cipriany B. R., Zhao R., Murphy P. J., Levy S. L., Tan C. P., Craighead H. G. and Soloway P. D. (2010) Single Molecule Epigenetic Analysis in a Nanofluidic Channel. Analytical Chemistry, 82, 2480-2487. First published on, doi: 10.1021/ac9028642.
Cunningham J M, Christensen E R, Tester D J, et al. Hypermethylation of the hMLH1 promoter in colon cancer with microsatellite instability. Cancer Res. 1998; 58(15):3455-60. Available at: http://www.ncbi.nlm.nih.gov/pubmed/9699680. Accessed Jan. 27, 2016.
Feinberg A. P. (2007) Phenotypic plasticity and the epigenetics of human disease. Nature, 447, 433-440. First published on.
Fraga M. F., Ballestar E., Montoya G., Taysavang P., Wade P. A. and Esteller M. (2003) The affinity of different MBD proteins for a specific methylated locus depends on their intrinsic binding properties. Nucleic Acids Research, 31, 1765-1774. First published on, doi: 10.1093/nar/gkg249.
Gall A, Hoffmann B, Harder T, Grund C, Hoper D, Beer M. Design and validation of a microarray for detection, hemagglutinin subtyping, and pathotyping of avian influenza viruses. J Clin Microbiol. 2009; 47(2):327-34. doi: 10.1128/JCM.01330-08.
Gebhard C, Schwarzfischer L, Pham T-H, et al. Genome-wide profiling of CpG methylation identifies novel targets of aberrant hypermethylation in myeloid leukemia. Cancer Res. 2006; 66(12):6118-28. doi:10.1158/0008-5472.CAN-06-0376.
Genereux D. P., Johnson W. C., Burden A. F., Stoger R. and Laird C. D. (2008) Errors in the bisulfite conversion of DNA: modulating inappropriate- and failed-conversion frequencies. Nucleic Acids Research, 36, e150. First published on, doi: 10.1093/nar/gkn691.
Ghosh R. P., Horowitz-Scherer R. A., Nikitina T., Gierasch L. M. and Woodcock C. L. (2008) Rett Syndrome-causing Mutations in Human MeCP2 Result in Diverse Structural Changes That Impact Folding and DNA Interactions. Journal of Biological Chemistry, 283, 20523-20534. First published on, doi: 10.1074/jbc.M803021200.
Gitan R S, Shi H, Chen C-M, Yan P S, Huang T H-M. Methylation-specific oligonucleotide microarray: a new potential for high-throughput methylation analysis. Genome Res. 2002; 12(1):158-64. doi:10.1101/gr.202801.
Grunau C., Clark S. J. and Rosenthal A. (2001) Bisulfite genomic sequencing: systematic investigation of critical experimental parameters. Nucleic Acids Research, 29, e65. First published on, doi: 10.1093/nar/29.13.e65.
Hashimshony T., Zhang J., Keshet I., Bustin M. and Cedar H. (2003) The role of DNA methylation in setting up chromatin structure during development. Nat Genet, 34, 187-192. First published on.
Hegi M E, Diserens A C, Godard S, et al. Clinical Trial Substantiates the Predictive Value of O-6-Methylguanine-DNA Methyltransferase Promoter Methylation in Glioblastoma Patients Treated with Temozolomide. Clin Cancer Res. 2004; 10(21): 1871-1874. doi:10.1158/1078-0432.CCR-03-0384.
Hegi M. E., Diserens A.-C., Gorlia T., Hamou M.-F., de Tribolet N., Weller M., Kros J. M., Hainfellner J. A., Mason W., Mariani L. et al. (2005) MGMT Gene Silencing and Benefit from Temozolomide in Glioblastoma. New England Journal of Medicine, 352, 997-1003. First published on, doi: doi:10.1056/NEJMoa043331.
Heimer B. W., Shatova T. A., Lee J. K., Kaastrup K. and Sikes H. D. (2014) Evaluating the sensitivity of hybridization-based epigenotyping using a methyl binding domain protein. Analyst, 139, 3695-3701. First published on, doi: 10.1039/c4an00667d.
Heimer B W, Tam B E, Sikes H D. Characterization and directed evolution of a methyl-binding domain protein for high-sensitivity DNA methylation analysis. Protein Eng Des Sel. 2015; 28(12):543-51. doi:10.1093/protein/gzv046.
Heimer B W, Tam B E, Minkovsky A, Sikes H D. Using nanobiotechnology to increase the prevalence of epigenotyping assays in precision medicine. Wiley Interdiscip Rev Nanomed Nanobiotechnol. 2016. doi: 10.1002/wnan. 1407.
Hendrich B. and Bird A. (1998) Identification and Characterization of a Family of Mammalian Methyl-CpG Binding Proteins. Mol Cell Biol, 18, 6538-6547. First published on.
Hendrich B., Hardeland U., Ng H.-H., Jiricny J. and Bird A. (1999) The thymine glycosylase MBD4 can bind to the product of deamination at methylated CpG sites. Nature, 401, 301-304. First published on.
Herman J G, Graff J R, Myohitnen S, Nelkin B D, Baylin S B. Methylation-specific PCR: a novel PCR assay for methylation status of CpG islands. Proc Natl Acad Sci USA. 1996; 93(18):9821-9826. Available at: http://www.pubmedcentral.nih.gov/articlerender.fcgi?artid=38513&tool=pmcentrez&rendertype=abstract.
Herman J. G., Umar A., Polyak K., Graff J. R., Ahuja N., Issa J.-P. J., Markowitz S., Willson J. K. V., Hamilton S. R., Kinzler K. W. et al. (1998) Incidence and functional consequences of hMLH1 promoter hypermethylation in colorectal carcinoma. Proceedings of the National Academy of Sciences, 95, 6870-6875. First published on.
Heyn H. and Esteller M. (2012) DNA methylation profiling in the clinic: applications and challenges. Nat Rev Genet, 13, 679-692. First published on.
Ho K. L., McNae I. W., Schmiedeberg L., Klose R. J., Bird A. P. and Walkinshaw M. D. (2008) MeCP2 Binding to DNA Depends upon Hydration at Methyl-CpG. Molecular cell, 29, 525-531. First published on.
Imperiale T F, RansohoffDF, Itzkowitz S H, et al. Multitarget stool DNA testing for colorectal-cancer screening. N Engl J Med. 2014; 370(14): 1287-97. doi:10.1056/NEJMoa1311194.
Jorgensen H. F., Adie K., Chaubert P. and Bird A. P. (2006) Engineering a high-affinity methyl-CpG-binding protein. Nucleic Acids Research, 34, e96. First published on, doi: 10.1093/nar/gk1527.
Kaastrup K., Chan L. and Sikes H. D. (2013) Impact of Dissociation Constant on the Detection Sensitivity of Polymerization-Based Signal Amplification Reactions. Analytical Chemistry, 85, 8055-8060. First published on, doi: 10.1021/ac4018988.
Laird P. W. (2010) Principles and challenges of genome-wide DNA methylation analysis. Nat Rev Genet, 11, 191-203. First published on.
Lipov\check{s}ek D., Lippow S. M., Hackel B. J., Gregson M. W., Cheng P., Kapila A. and Wittrup K. D. (2007) Evolution of an Interloop Disulfide Bond in High-Affinity Antibody Mimics Based on Fibronectin Type III Domain and Selected by Yeast Surface Display: Molecular Convergence with Single-Domain Camelid and Shark Antibodies. Journal of Molecular Biology, 368, 1024-1041. First published on, doi: http://dx.doi.org/10.1016/j.jmb.2007.02.029.
Luo J., Zheng W., Wang Y., Wu Z., Bai Y. and Lu Z. (2009) Detection method for methylation density on microarray using methyl-CpG-binding domain protein. Analytical Biochemistry, 387, 143-149. First published on, doi: http://dx.doi.org/10.1016/j.ab.2008.11.020.
Nan X., Meehan R. R. and Bird A. (1993) Dissection of the methyl-CpG binding domain from the chromosomal protein MeCP2. Nucleic Acids Research, 21, 4886-4892. First published on, doi: 10.1093/nar/21.21.4886.
Noehammer C, Pulverer W, Hassler M R, et al. Strategies for validation and testing of DNA methylation biomarkers. Epigenomics. 2014; 6(6):603-22. doi:10.2217/epi.14.43.
Ohki I., Shimotake N., Fujita N., Jee J.-G., Ikegami T., Nakao M. and Shirakawa M. (2001) Solution Structure of the Methyl-CpG Binding Domain of Human MBD1 in Complex with Methylated DNA. Cell, 105, 487-497. First published on, doi: 10.1016/s0092-8674(01)00324-5.
Okamoto A. Chemical approach toward efficient DNA methylation analysis. Org Biomol Chem. 2009; 7(1):21-26. doi:10.1039/B813595A.
Pomraning K. R., Smith K. M. and Freitag M. (2009) Genome-wide high throughput analysis of DNA methylation in eukaryotes. Methods, 47, 142-150. First published on, doi: http://dx.doi.org/10.1016/j.ymeth.2008.09.022.
Porter J. R., Stains C. I., Segal D. J. and Ghosh I. (2007) Split β-Lactamase Sensor for the Sequence-Specific Detection of DNA Methylation. Analytical Chemistry, 79, 6702-6708. First published on, doi: 10.1021/ac071163+.
Potter N T, Hurban P, White M N, et al. Validation of a Real-Time PCR-Based Qualitative Assay for the Detection of Methylated SEPT9 DNA in Human Plasma. Clin Chem. 2014; 000: 1-9. doi: 10.1373/clinchem.2013.221044.
Pratt V M. Are we ready for a blood-based test to detect colon cancer? Clin Chem. 2014; 60(9): 1141-2. doi: 10.1373/clinchem.2014.227132.
Roadmap Epigenomics C., Kundaje A., Meuleman W., Ernst J., Bilenky M., Yen A., Heravi-Moussavi A., Kheradpour P., Zhang Z., Wang J. et al. (2015) Integrative analysis of 111 reference human epigenomes. Nature, 518, 317-330. First published on, doi: 10.1038/nature 14248 http://www.nature.com/nature/j ournal/v518/n7539/abs/nature 14248.html#supplementary-information.
Scarsdale J. N., Webb H. D., Ginder G. D. and Williams D. C. (2011) Solution structure and dynamic analysis of chicken MBD2 methyl binding domain bound to a target-methylated DNA sequence. Nucleic Acids Research. First published on, doi: 10.1093/nar/gkr262.
Shapiro E., Biezuner T. and Linnarsson S. (2013) Single-cell sequencing-based technologies will revolutionize whole-organism science. Nat Rev Genet, 14, 618-630. First published on, doi: 10.1038/nrg3542.
Shusta E. V., Kieke M. C., Parke E., Kranz D. M. and Wittrup K. D. (1999) Yeast polypeptide fusion surface display levels predict thermal stability and soluble secretion efficiency. Journal of Molecular Biology, 292, 949-956. First published on, doi: http://dx.doi.org/10.1006/jmbi.1999.3130.
Silver D. P., Richardson A. L., Eklund A. C., Wang Z. C., Szallasi Z., Li Q., Juul N., Leong C.-O., Calogrias D., Buraimoh A. et al. (2010) Efficacy of Neoadjuvant Cisplatin in Triple-Negative Breast Cancer. Journal of Clinical Oncology, 28, 1145-1153. First published on, doi: 10.1200/jco.2009.22.4725.
Van Antwerp J. J. and Wittrup K. D. (2000) Fine Affinity Discrimination by Yeast Surface Display and Flow Cytometry. Biotechnology Progress, 16, 31-37. First published on, doi: 10.1021/bp990133s.
Van Neste L., Herman J. G., Otto G., Bigley J. W., Epstein J. I. and Van Criekinge W. (2012) The Epigenetic promise for prostate cancer diagnosis. The Prostate, 72, 1248-1261. First published on, doi: 10.1002/pros.22459.
Veigl M L, Kasturi L, Olechnowicz J, et al. Biallelic inactivation of hMLH1 by epigenetic gene silencing, a novel mechanism causing human MSI cancers. Proc Natl Acad Sci. 1998; 95(15):8698-8702. doi:10.1073/pnas.95.15.8698.
Waldmuller S, Freund P, Mauch S, Toder R, Vosberg H-P. Low-density DNA microarrays are versatile tools to screen for known mutations in hypertrophic cardiomyopathy. Hum Mutat. 2002; 19(5):560-9. doi:10.1002/humu. 10074.
Wang D. and Bodovitz S. (2010) Single cell analysis: the new frontier in ‘omics’. Trends in Biotechnology, 28, 281-290. First published on, doi: http://dx.doi.org/10.1016/j.tibtech.2010.03.002.
Wolffe A. P. and Matzke M. A. (1999) Epigenetics: Regulation Through Repression. Science, 286, 481-486. First published on, doi: 10.1126/science.286.5439.481.
Yu Y., Blair S., Gillespie D., Jensen R., Myszka D., Badran A. H., Ghosh I. and Chagovetz A. (2010) Direct DNA Methylation Profiling Using Methyl Binding Domain Proteins. Analytical Chemistry, 82, 5012-5019. First published on, doi: 10.1021/ac1010316.

1. An isolated hMBD2 nucleic acid sequence comprising a sequence selected from the group consisting of:
a) a nucleic acid selected from:
(SEQ ID NO: 33)
GAAAGCGGCAAACGCATGGATTGCCCGGCGCTGCCGCCGGGTTGGAA
AAGAGAAGAAGTGATTCGTAAAAGCGGCCTGAGCGCGGGCAAAAGCG
ATGTGTATTATTTTAGCCCGAGCGGCAAAAAAATTCGTAGCAAACCGC
AGCTGGCGCGTTATCTGGGCAACTCCGTGGATCTGAGCAGCTTTGATT
ATCGTACCGGCAAAATG;
(SEQ ID NO: 1)
GAAAGCGGCAAACGCATGGATTGCCCGGCGCTGCCGCCGGGTTGGAA
AAGAGAAGAAGTGATTCGTAAAAGCGGCCGGAGCGCGGGCAAAATCG
ATGTGTATTATTTTAGCCCGAGCGGCAAAAAAATTCGTAGCAAACCGC
AGCTGGCGCGTTATCTGGGCAACACCGTGGATCTGAGCAGCTTTGATT
ATCGTACCGGCAAAATG;
(SEQ ID NO: 27)
GAAAGCGGCAAACGCATGGATTGCCCGGCGCTGCCGCCGGGTTGGAA
AAGAGAAGAAGTGATTCGTAAAAGCGGCCTGAGCGCGGGCAAAAGCG
ATGTGTATTATTTTAGCCCGAGCGGCAAAAAATTTCGTAGCAAACCGC
AGCTGGCGCGTTATCTGGGCAACACCGTGGATCTGAGCAGCTTTGATT
TTCGTACCGGCAAAATG;
(SEQ ID NO: 28)
GAAAGCGGCAAACGCATGGATTGCCCGGCGCTGCCGCCGGGTTGGAA
AAGAGAAGAAGTGATTCGTAAAAGCGGCCTGAGCGCGGGCAAAAGCG
ATGTGTATTATTTTAGCCCGAGCGGCAAAAAATTTCGTAGCAAACCGC
AGCTGGCGCGTTATCTGGGCAACACCGTGGATCTGAGCAGCTTTGATT
ATCGTACCGGCAAAATG;
(SEQ ID NO: 29)
GAAAGCGGCAAACGCATGGATTGCCCGGCGCTGCCGCCGGGTTGGAA
AAGAGAAGAAGTGATTCGTAAAAGCGGCCTGAGCGCGGGCAAAATCG
ATGTGTATTATTTTAGCCCGAGCGGCAAAAAATTTCGTAGCAAACCGC
AGCTGGCGCGTTATCTGGGCAACACCGTGGATCTGAGCAGCTTTGATT
TTCGTACCGGCAAAATG;
(SEQ ID NO: 30)
GAAAGCGGCAAACGCACGGATTGCCCGGCGCTGCCGCCGGGTTGGAA
AAGAGAAGAAGTGATTCGTAAAAGCGGCCTGAGCGCGGGCAAAAGCG
ATGTGTATTATTTTAGCCCGAGCGGCAAAAAATTTCGTAGCAAACCGC
AGCTGGCGCGTTATCTGGGCAACACCGTGGATCTGAGCAGCTTTGATT
TTCGTACCGGCAAAATG;
(SEQ ID NO: 31)
GAAAGCGGCAAACGCATGGATTGCCCGGCGCTGCCGCCGGGTTGGAA
AAGAGAAGAAGTGATTCGTAAAAGCGGCCTGAGCGCGGGCAAAAGCG
ATGTGTATTATTTTAGCCCGAGCGGCAAAAAATTTCGTAGCAAACGGC
AGCTGGCGCGTTATCTGGGCAACACCGTGGATCTGAGCAGCTTTGATT
ATCGTACCGGCAAAATG;
(SEQ ID NO: 32)
GAAAGCGGCAAACGCATGGATTGCCCGGCGCTGCCGCCGGGTTGGAA
AAGAGAAGAAGTGATTCGTAAAAGCGGCCTGAGCGCGGGCAAAATCG
ATGTGTATTATTTTAGCCCGAGCGGCAAAAAAATTCGTAGCAAACCGC
AGCTGGCGCGTTATCTGGGCAACACCGTGGATCTGAGCAGCTTTGATT
TTCGTACCTGCAAAATG;
(SEQ ID NO: 34)
GAAAGCGGCAAACGCATGGATTGCCCGGCGCTGCCGCCGGGTTGGAA
AAGAGAAGAAGTGATTCGTAAAAGCGGCCTGAGCGCGGGCAAAAGCG
ATGTGTATTATTATAGCCCGAGCGGCAAAAAATTTCGTAGCAAACCGC
AGCTGGCGCGTTATCTGGGCAACACCGTGGATCTGAGCAGCTTTGATT
ATCGTACCGGCAAAATG;
(SEQ ID NO: 35)
GAAAGCGGCAAACGCATGGATTGCCCGGCGCTGCCGCCGGGTTGGAA
AAGAGAAGAAGTGATCCGTAAAAGCGGCCTGAGCGCGGGCAAAAGCG
ATGTGTATTATTTTAGCCCGAGCGGCAAAAAAATTCGTAGCAAACCGC
AGCTGGCGCGTTATCTGGGCAACACCGTGGATCTGAGCAGCTTTGATT
ATCGTACCGGCAAAATG;
or
(SEQ ID NO: 36)
GAAAGCGGCAAACGCATGGATTGCCCGGCGCTGCCGCCGGGTTGGAA
AAGAGAAGAAGTGATTCGTAAAAGCGGCCTGAGCGCGGGCAAAATCG
ATGTGTATTATTTTAGCCCGAGCGGCAAAAAAATTCGTAGCAAACCGC
AGCTGGCGCGTTATCTGGGCAACACCGTGGATCTGAGCAGCTTTGATT
ATCGTACCGGCAAAATG;
b) a sequence which specifically hybridizes with the full length sequence of SEQ ID NO: 33; SEQ ID NO: 1, SEQ ID NO: 27; SEQ ID NO: 28; SEQ ID NO: 29; SEQ ID NO: 30; SEQ ID NO: 31, SEQ ID NO: 32; SEQ ID NO: 34; SEQ ID NO: 35; or SEQ ID NO: 36;
c) a sequence encoding the polypeptide comprising an amino acid sequence selected from:
(SEQ ID NO: 23)
ESGKRMDCPALPPGWKREEVIRKSGLSAGKSDVYYFSPSGKKIRSKPQLA
RYLGNSVDLSSFDYRTGKM;
(SEQ ID NO: 14)
ESGKRMDCPALPPGWKREEVIRKSGRSAGKIDVYYFSPSGKKIRSKPQLA
RYLGNTVDLSSFDYRTGKM;
(SEQ ID NO: 7)
ESGKRMDCPALPPGWKKEVVIRKSGLSAGKSDVYYFSPSGKKFRSKPQL
ARYLGNTVDLSSFDYRTGKM;
(SEQ ID NO: 8)
ESGKRMDCPALPPGWKREEVIRKSGLSAGKSDVYYFSPSGKKFRSKPQLA
RYLGNTVDLSSFDFRTGKM;
(SEQ ID NO: 9)
ESGKRMDCPALPPGWKREEVIRKSGLSAGKSDVYYFSPSGKKFRSKPQLA
RYLGNTVDLSSFDYRTGKM;
(SEQ ID NO: 10)
ESGKRMDCPALPPGWKKEEVIRKSGLSAGKIDVYYFSPSGKKIRSKPQLA
RYLGNTVDLSSFDFRTGKM;
(SEQ ID NO: 11)
ESGKRMDCPALPPGWKREEVIRKSGLSAGKRDVYYFSPSGKKFRSKPQLA
RYLGNTVDLSSFDFRTGKM;
(SEQ ID NO: 12)
ESGKRMDCPALPPGWKREEVIRKSGLSAGKIDVYYFSPSGKKFRSKPQLA
RYLGNTVDLSSFDFRTGKM;
(SEQ ID NO: 13)
ESGKRTDCPALPPGWKREEVIRKSGLSAGKSDVYYFSPSGKKFRSKPQLA
RYLGNTVDLSSFDFRTGKM;
(SEQ ID NO: 15)
ESGKRMDCPALPPGWKREEVIRKSGLSAGKSDVYYFSPSGKKFRSKRQLA
RYLGNTVDLSSFDYRTGKM;
(SEQ ID NO: 22)
ESGKRMDCPALPPGWKREEVIRKSGLSAGKIDVYYFSPSGKKIRSKPQLA
RYLGNTVDLSSFDFRTCKM;
(SEQ ID NO: 24)
ESGKRMDCPALPPGWKREEVIRKSGLSAGKSDVYYYSPSGKKFRSKPQL
ARYLGNTVDLSSFDYRTGKM;
(SEQ ID NO: 25)
ESGKRMDCPALPPGWKREEVIRKSGLSAGKSDVYYFSPSGKKIRSKPQLA
RYLGNTVDLSSFDYRTGKM;
or
(SEQ ID NO: 26)
ESGKRMDCPALPPGWKREEVIRKSGLSAGKIDVYYFSPSGKKIRSKPQLA
RYLGNTVDLSSFDYRTGKM;
and
d) conservatively modified variants thereof.

2. A polypeptide comprising the amino acid sequence selected from:
(SEQ ID NO: 23)
ESGKRMDCPALPPGWKREEVIRKSGLSAGKSDVYYFSPSGKKIRSKPQLA
RYLGNSVDLSSFDYRTGKM;
(SEQ ID NO: 14)
ESGKRMDCPALPPGWKREEVIRKSGRSAGKIDVYYFSPSGKKIRSKPQLA
RYLGNTVDLSSFDYRTGKM;
(SEQ ID NO: 7)
ESGKRMDCPALPPGWKKEVVIRKSGLSAGKSDVYYFSPSGKKFRSKPQL
ARYLGNTVDLSSFDYRTGKM;
(SEQ ID NO: 8)
ESGKRMDCPALPPGWKREEVIRKSGLSAGKSDVYYFSPSGKKFRSKPQLA
RYLGNTVDLSSFDFRTGKM;
(SEQ ID NO: 9)
ESGKRMDCPALPPGWKREEVIRKSGLSAGKSDVYYFSPSGKKFRSKPQLA
RYLGNTVDLSSFDYRTGKM;
(SEQ ID NO: 10)
ESGKRMDCPALPPGWKKEEVIRKSGLSAGKIDVYYFSPSGKKIRSKPQLA
RYLGNTVDLSSFDFRTGKM;
(SEQ ID NO: 11)
ESGKRMDCPALPPGWKREEVIRKSGLSAGKRDVYYFSPSGKKFRSKPQLA
RYLGNTVDLSSFDFRTGKM;
(SEQ ID NO: 12)
ESGKRMDCPALPPGWKREEVIRKSGLSAGKIDVYYFSPSGKKFRSKPQLA
RYLGNTVDLSSFDFRTGKM;
(SEQ ID NO: 13)
ESGKRTDCPALPPGWKREEVIRKSGLSAGKSDVYYFSPSGKKFRSKPQLA
RYLGNTVDLSSFDFRTGKM;
(SEQ ID NO: 15)
ESGKRMDCPALPPGWKREEVIRKSGLSAGKSDVYYFSPSGKKFRSKRQLA
RYLGNTVDLSSFDYRTGKM;
(SEQ ID NO: 22)
ESGKRMDCPALPPGWKREEVIRKSGLSAGKIDVYYFSPSGKKIRSKPQLA
RYLGNTVDLSSFDFRTCKM;
(SEQ ID NO: 24)
ESGKRMDCPALPPGWKREEVIRKSGLSAGKSDVYYYSPSGKKFRSKPQL
ARYLGNTVDLSSFDYRTGKM;
(SEQ ID NO: 25)
ESGKRMDCPALPPGWKREEVIRKSGLSAGKSDVYYFSPSGKKIRSKPQLA
RYLGNTVDLSSFDYRTGKM;
or
(SEQ ID NO: 26)
ESGKRMDCPALPPGWKREEVIRKSGLSAGKIDVYYFSPSGKKIRSKPQLA
RYLGNTVDLSSFDYRTGKM;

3. A conservatively modified variant of the polypeptide according to claim 2, wherein the conservatively modified polypeptide binds a DNA sequence having a single methylated CpG site with a dissociation constant (Kd) greater than or equal to 3.1±1.0 nM.

4. A protein for detecting methylated CpG (mCpG) comprising the polypeptide according to claim 2.

5. A protein for detecting methylated CpG (mCpG) comprising the polypeptide according to claim 3.

6. A fusion protein comprising the polypeptide according to claim 2 and a reporter protein.

7. A vector comprising the nucleic acid molecule of claim 1.

8. The vector of claim 7, wherein the nucleic acid molecule is operatively linked to an expression control sequence allowing expression in prokaryotic or eukaryotic host cells.

9. A polypeptide having the amino acid sequence encoded by the nucleic acid molecule of claim 1.

10. A composition comprising the nucleic acid molecule of claim 1.

11. The composition of claim 10 which is a diagnostic composition optionally further comprising suitable diagnostic means.

12. A method for detecting methylated CpG DNA in a sample, the method comprising obtaining a sample; contacting the sample with a fusion protein according to claim 6; and detecting the binding of said protein to methylated DNA.

13. An in vitro method for detecting methylated DNA in a sample comprising
(a) contacting a sample with the polypeptide of claim 10; and
(b) detecting the binding of the polypeptide of claim 10 to methylated DNA.

14. An in vitro method for detecting methylated DNA in a sample comprising
(a) contacting a sample with the polypeptide of claim 2; and
(b) detecting the binding of the polypeptide of claim 2 to methylated DNA.

15. An in vitro method for detecting methylated DNA in a sample comprising
(a) contacting a sample with the fusion protein of claim 6; and
(b) detecting the binding of the fusion protein of claim 6 to methylated DNA.

16. An isolated hMBD2 nucleic acid sequence comprising a sequence selected from the group consisting of:
a) SEQ ID NO: 33;
b) the sequence which specifically hybridizes with the full length sequence of SEQ ID NO: 33;
c) the sequence encoding the polypeptide comprising ESGKRMDCPALPPGWKREEVIRKSGRSAGKIDVYYFSPSGKKIRSKPQLA RYLGNTVDLSSFDYRTGKM (SEQ ID NO: 23); and
d) conservatively modified variants thereof.

17. A polypeptide comprising the amino acid sequence ESGKRMDCPALPPGWKREEVIRKSGLSAGKSDVYYFSPSGKKIRSKPQLA RYLGNSVDLSSFDYRTGKM (SEQ ID NO: 23).

18. A conservatively modified variant of the polypeptide according to claim 17, wherein the conservatively modified polypeptide binds a DNA sequence having a single methylated CpG site with a dissociation constant (Kd) greater than or equal to 3.1±1.0 nM.

19. A protein for detecting methylated CpG (mCpG) comprising the polypeptide according to claim 17.

20. A protein for detecting methylated CpG (mCpG) comprising the polypeptide according to claim 18.

21. A fusion protein comprising the polypeptide according to claim 17 and a reporter protein.

22. A vector comprising the nucleic acid molecule of claim 16.

23. The vector of claim 22, wherein the nucleic acid molecule is operatively linked to an expression control sequence allowing expression in prokaryotic or eukaryotic host cells.

24. A polypeptide having the amino acid sequence encoded by the nucleic acid molecule of claim 16.

25. A composition comprising the nucleic acid molecule of claim 16.

26. The composition of claim 25 which is a diagnostic composition optionally further comprising suitable diagnostic means.

27. A method for detecting methylated CpG DNA in a sample, the method comprising obtaining a sample; contacting the sample with a fusion protein according to claim 21; and detecting the binding of said protein to methylated DNA.

28. An in vitro method for detecting methylated DNA in a sample comprising
(a) contacting a sample with the polypeptide of claim 24; and
(b) detecting the binding of the polypeptide of claim 24 to methylated DNA.

29. An in vitro method for detecting methylated DNA in a sample comprising
(a) contacting a sample with the polypeptide of claim 17; and
(b) detecting the binding of the polypeptide of claim 17 to methylated DNA.

30. An in vitro method for detecting methylated DNA in a sample comprising
(a) contacting a sample with the fusion protein of claim 21; and
(b) detecting the binding of the fusion protein of claim 21 to methylated DNA.

31. An isolated hMBD2 nucleic acid sequence comprising a sequence selected from the group consisting of:
a) SEQ ID NO: 1;
b) the sequence which specifically hybridizes with the full length sequence of SEQ ID NO: 1;
c) the sequence encoding the polypeptide comprising ESGKRMDCPALPPGWKREEVIRKSGRSAGKIDVYYFSPSGKKIRSKPQLA RYLGNTVDLSSFDYRTGKM (SEQ ID NO: 14); and
d) conservatively modified variants thereof.

32. A polypeptide comprising the amino acid sequence ESGKRMDCPALPPGWKREEVIRKSGRSAGKIDVYYFSPSGKKIRSKPQLA RYLGNTVDLSSFDYRTGKM (SEQ ID NO: 14).

33. A conservatively modified variant of the polypeptide according to claim 32, wherein the conservatively modified polypeptide binds a DNA sequence having a single methylated CpG site with a dissociation constant (Kd) greater than or equal to 3.1±1.0 nM.

34. A protein for detecting methylated CpG (mCpG) comprising the polypeptide according to claim 32.

35. A protein for detecting methylated CpG (mCpG) comprising the polypeptide according to claim 33.

36. A fusion protein comprising the polypeptide according to claim 32 and a reporter protein.

37. A vector comprising the nucleic acid molecule of claim 31.

38. The vector of claim 37, wherein the nucleic acid molecule is operatively linked to an expression control sequence allowing expression in prokaryotic or eukaryotic host cells.

39. A polypeptide having the amino acid sequence encoded by the nucleic acid molecule of claim 31.

40. A composition comprising the nucleic acid molecule of claim 31.

41. The composition of claim 40 which is a diagnostic composition optionally further comprising suitable diagnostic means.

42. A method for detecting methylated CpG DNA in a sample, the method comprising obtaining a sample; contacting the sample with a fusion protein according to claim 36; and detecting the binding of said protein to methylated DNA.

43. An in vitro method for detecting methylated DNA in a sample comprising
(a) contacting a sample with the polypeptide of claim 40; and
(b) detecting the binding of the polypeptide of claim 40 to methylated DNA.

44. An in vitro method for detecting methylated DNA in a sample comprising
(a) contacting a sample with the polypeptide of claim 32; and
(b) detecting the binding of the polypeptide of claim 32 to methylated DNA.

45. An in vitro method for detecting methylated DNA in a sample comprising
(a) contacting a sample with the fusion protein of claim 36; and
(b) detecting the binding of the fusion protein of claim 36 to methylated DNA.


SEQUENCE LISTING (52 sequences)

SEQ ID NO: 1 - DNA, 207 bp, Artificial Sequence
  Description of Artificial Sequence Synthetic polynucleotide
gaaagcggcaaacgcatggattgcccggcgctgccgccgggttggaaaagagaagaagtgattcgtaaaagcggccggagcgcgggcaaaatcgatgtgtattattttagcccgagcggcaaaaaaattcgtagcaaaccgcagctggcgcgttatctgggcaacaccgtggatctgagcagctttgattatcgtaccggcaaaatg

SEQ ID NO: 2 - DNA, 22 bp, Artificial Sequence
  Description of Artificial Sequence Synthetic oligonucleotide
tttgcggtccgctgcccgaccc

SEQ ID NO: 3 - DNA, 22 bp, Artificial Sequence
  Description of Artificial Sequence Synthetic oligonucleotide
gggtcgggcagcggaccgcaaa

SEQ ID NO: 4 - DNA, 333 bp, Artificial Sequence
  Description of Artificial Sequence Synthetic polynucleotide
tacccatacgacgttccagactacgctctgcaggctagtggtggaggaggctctggtggaggcggtagcggaggcggagggtcggctagcgaaagcggcaaacgcatggattgcccggcgctgccgccgggttggaaaaaagaagaagtgattcgtaaaagcggcctgagcgcgggcaaaagcgatgtgtattattttagcccgagcggcaaaaaatttcgtagcaaaccgcagctggcgcgttatctgggcaacaccgtggatctgagcagctttgattttcgtaccggcaaaatgggatccgaacaaaagcttatttctgaagaggacttg

SEQ ID NO: 5 - protein, 111 aa, Artificial Sequence
  Description of Artificial Sequence Synthetic polypeptide
YPYDVPDYALQASGGGGSGGGGSGGGGSASESGKRMDCPALPPGWKKEEVIRKSGLSAGKSDVYYFSPSGKKFRSKPQLARYLGNTVDLSSFDFRTGKMGSEQKLISEEDL

SEQ ID NO: 6 - protein, 69 aa, Homo sapiens
ESGKRMDCPALPPGWKKEEVIRKSGLSAGKSDVYYFSPSGKKFRSKPQLARYLGNTVDLSSFDFRTGKM

SEQ ID NO: 7 - protein, 69 aa, Artificial Sequence
  Description of Artificial Sequence Synthetic polypeptide
ESGKRMDCPALPPGWKKEVVIRKSGLSAGKSDVYYFSPSGKKFRSKPQLARYLGNTVDLSSFDYRTGKM

SEQ ID NO: 8 - protein, 69 aa, Artificial Sequence
  Description of Artificial Sequence Synthetic polypeptide
ESGKRMDCPALPPGWKREEVIRKSGLSAGKSDVYYFSPSGKKFRSKPQLARYLGNTVDLSSFDFRTGKM

SEQ ID NO: 9 - protein, 69 aa, Artificial Sequence
  Description of Artificial Sequence Synthetic polypeptide
ESGKRMDCPALPPGWKREEVIRKSGLSAGKSDVYYFSPSGKKFRSKPQLARYLGNTVDLSSFDYRTGKM

SEQ ID NO: 10 - protein, 69 aa, Artificial Sequence
  Description of Artificial Sequence Synthetic polypeptide
ESGKRMDCPALPPGWKKEEVIRKSGLSAGKIDVYYFSPSGKKIRSKPQLARYLGNTVDLSSFDFRTGKM

SEQ ID NO: 11 - protein, 69 aa, Artificial Sequence
  Description of Artificial Sequence Synthetic polypeptide
ESGKRMDCPALPPGWKREEVIRKSGLSAGKRDVYYFSPSGKKFRSKPQLARYLGNTVDLSSFDFRTGKM

SEQ ID NO: 12 - protein, 69 aa, Artificial Sequence
  Description of Artificial Sequence Synthetic polypeptide
ESGKRMDCPALPPGWKREEVIRKSGLSAGKIDVYYFSPSGKKFRSKPQLARYLGNTVDLSSFDFRTGKM

SEQ ID NO: 13 - protein, 69 aa, Artificial Sequence
  Description of Artificial Sequence Synthetic polypeptide
ESGKRTDCPALPPGWKREEVIRKSGLSAGKSDVYYFSPSGKKFRSKPQLARYLGNTVDLSSFDFRTGKM

SEQ ID NO: 14 - protein, 69 aa, Artificial Sequence
  Description of Artificial Sequence Synthetic polypeptide
ESGKRMDCPALPPGWKREEVIRKSGRSAGKIDVYYFSPSGKKIRSKPQLARYLGNTVDLSSFDYRTGKM

SEQ ID NO: 15 - protein, 69 aa, Artificial Sequence
  Description of Artificial Sequence Synthetic polypeptide
ESGKRMDCPALPPGWKREEVIRKSGLSAGKSDVYYFSPSGKKFRSKRQLARYLGNTVDLSSFDYRTGKM

SEQ ID NO: 16 - DNA, 207 bp, Homo sapiens
gaaagcggcaaacgcatggattgcccggcgctgccgccgggttggaaaaaagaagaagtgattcgtaaaagcggcctgagcgcgggcaaaagcgatgtgtattattttagcccgagcggcaaaaaatttcgtagcaaaccgcagctggcgcgttatctgggcaacaccgtggatctgagcagctttgattttcgtaccggcaaaatg

SEQ ID NO: 17 - DNA, 24 bp, Artificial Sequence
  Description of Artificial Sequence Synthetic primer
tacagctagcgaaagcggcaaacg

SEQ ID NO: 18 - DNA, 26 bp, Artificial Sequence
  Description of Artificial Sequence Synthetic primer
gacaggatcccattttgccggtacga

SEQ ID NO: 19 - DNA, 50 bp, Artificial Sequence
  Description of Artificial Sequence Synthetic primer
cgacgattgaaggtagatacccatacgacgttccagactacgctctgcag

SEQ ID NO: 20 - DNA, 47 bp, Artificial Sequence
  Description of Artificial Sequence Synthetic primer
cagatctcgagctattacaagtcctcttcagaaataagcttttgttc

SEQ ID NO: 21 - DNA, 21 bp, Artificial Sequence
  Description of Artificial Sequence Synthetic primer
cccctcaactagcaaaggcag

SEQ ID NO: 22 - protein, 69 aa, Artificial Sequence
  Description of Artificial Sequence Synthetic polypeptide
ESGKRMDCPALPPGWKREEVIRKSGLSAGKIDVYYFSPSGKKIRSKPQLARYLGNTVDLSSFDFRTCKM

SEQ ID NO: 23 - protein, 69 aa, Artificial Sequence
  Description of Artificial Sequence Synthetic polypeptide
ESGKRMDCPALPPGWKREEVIRKSGLSAGKSDVYYFSPSGKKIRSKPQLARYLGNSVDLSSFDYRTGKM

SEQ ID NO: 24 - protein, 69 aa, Artificial Sequence
  Description of Artificial Sequence Synthetic polypeptide
ESGKRMDCPALPPGWKREEVIRKSGLSAGKSDVYYYSPSGKKFRSKPQLARYLGNTVDLSSFDYRTGKM

SEQ ID NO: 25 - protein, 69 aa, Artificial Sequence
  Description of Artificial Sequence Synthetic polypeptide
ESGKRMDCPALPPGWKREEVIRKSGLSAGKSDVYYFSPSGKKIRSKPQLARYLGNTVDLSSFDYRTGKM

SEQ ID NO: 26 - protein, 69 aa, Artificial Sequence
  Description of Artificial Sequence Synthetic polypeptide
ESGKRMDCPALPPGWKREEVIRKSGLSAGKIDVYYFSPSGKKIRSKPQLARYLGNTVDLSSFDYRTGKM

SEQ ID NO: 27 - DNA, 207 bp, Artificial Sequence
  Description of Artificial Sequence Synthetic polynucleotide
gaaagcggcaaacgcatggattgcccggcgctgccgccgggttggaaaagagaagaagtgattcgtaaaagcggcctgagcgcgggcaaaagcgatgtgtattattttagcccgagcggcaaaaaatttcgtagcaaaccgcagctggcgcgttatctgggcaacaccgtggatctgagcagctttgattttcgtaccggcaaaatg

SEQ ID NO: 28 - DNA, 207 bp, Artificial Sequence
  Description of Artificial Sequence Synthetic polynucleotide
gaaagcggcaaacgcatggattgcccggcgctgccgccgggttggaaaagagaagaagtgattcgtaaaagcggcctgagcgcgggcaaaagcgatgtgtattattttagcccgagcggcaaaaaatttcgtagcaaaccgcagctggcgcgttatctgggcaacaccgtggatctgagcagctttgattatcgtaccggcaaaatg

SEQ ID NO: 29 - DNA, 207 bp, Artificial Sequence
  Description of Artificial Sequence Synthetic polynucleotide
gaaagcggcaaacgcatggattgcccggcgctgccgccgggttggaaaagagaagaagtgattcgtaaaagcggcctgagcgcgggcaaaatcgatgtgtattattttagcccgagcggcaaaaaatttcgtagcaaaccgcagctggcgcgttatctgggcaacaccgtggatctgagcagctttgattttcgtaccggcaaaatg

SEQ ID NO: 30 - DNA, 207 bp, Artificial Sequence
  Description of Artificial Sequence Synthetic polynucleotide
gaaagcggcaaacgcacggattgcccggcgctgccgccgggttggaaaagagaagaagtgattcgtaaaagcggcctgagcgcgggcaaaagcgatgtgtattattttagcccgagcggcaaaaaatttcgtagcaaaccgcagctggcgcgttatctgggcaacaccgtggatctgagcagctttgattttcgtaccggcaaaatg

SEQ ID NO: 31 - DNA, 207 bp, Artificial Sequence
  Description of Artificial Sequence Synthetic polynucleotide
gaaagcggcaaacgcatggattgcccggcgctgccgccgggttggaaaagagaagaagtgattcgtaaaagcggcctgagcgcgggcaaaagcgatgtgtattattttagcccgagcggcaaaaaatttcgtagcaaacggcagctggcgcgttatctgggcaacaccgtggatctgagcagctttgattatcgtaccggcaaaatg

SEQ ID NO: 32 - DNA, 207 bp, Artificial Sequence
  Description of Artificial Sequence Synthetic polynucleotide
gaaagcggcaaacgcatggattgcccggcgctgccgccgggttggaaaagagaagaagtgattcgtaaaagcggcctgagcgcgggcaaaatcgatgtgtattattttagcccgagcggcaaaaaaattcgtagcaaaccgcagctggcgcgttatctgggcaacaccgtggatctgagcagctttgattttcgtacctgcaaaatg

SEQ ID NO: 33 - DNA, 207 bp, Artificial Sequence
  Description of Artificial Sequence Synthetic polynucleotide
gaaagcggcaaacgcatggattgcccggcgctgccgccgggttggaaaagagaagaagtgattcgtaaaagcggcctgagcgcgggcaaaagcgatgtgtattattttagcccgagcggcaaaaaaattcgtagcaaaccgcagctggcgcgttatctgggcaactccgtggatctgagcagctttgattatcgtaccggcaaaatg

SEQ ID NO: 34 - DNA, 207 bp, Artificial Sequence
  Description of Artificial Sequence Synthetic polynucleotide
gaaagcggcaaacgcatggattgcccggcgctgccgccgggttggaaaagagaagaagtgattcgtaaaagcggcctgagcgcgggcaaaagcgatgtgtattattatagcccgagcggcaaaaaatttcgtagcaaaccgcagctggcgcgttatctgggcaacaccgtggatctgagcagctttgattatcgtaccggcaaaatg

SEQ ID NO: 35 - DNA, 207 bp, Artificial Sequence
  Description of Artificial Sequence Synthetic polynucleotide
gaaagcggcaaacgcatggattgcccggcgctgccgccgggttggaaaagagaagaagtgatccgtaaaagcggcctgagcgcgggcaaaagcgatgtgtattattttagcccgagcggcaaaaaaattcgtagcaaaccgcagctggcgcgttatctgggcaacaccgtggatctgagcagctttgattatcgtaccggcaaaatg

SEQ ID NO: 36 - DNA, 207 bp, Artificial Sequence
  Description of Artificial Sequence Synthetic polynucleotide
gaaagcggcaaacgcatggattgcccggcgctgccgccgggttggaaaagagaagaagtgattcgtaaaagcggcctgagcgcgggcaaaatcgatgtgtattattttagcccgagcggcaaaaaaattcgtagcaaaccgcagctggcgcgttatctgggcaacaccgtggatctgagcagctttgattatcgtaccggcaaaatg

SEQ ID NO: 37 - protein, 6 aa, Artificial Sequence
  Description of Artificial Sequence Synthetic 6xHis tag
HHHHHH

SEQ ID NO: 38 - protein, 7 aa, Unknown
  Description of Unknown Nuclear localization sequence
PKKKRKV

SEQ ID NO: 39 - protein, 10 aa, Artificial Sequence
  Description of Artificial Sequence Synthetic peptide
GGGGSGGGGS

SEQ ID NO: 40 - DNA, 25 bp, Artificial Sequence
  Description of Artificial Sequence Synthetic primer
gcctgaattctgaaagcggcaaacg

SEQ ID NO: 41 - DNA, 31 bp, Artificial Sequence
  Description of Artificial Sequence Synthetic primer
cattttgccggtacgataatcaaagctgctc

SEQ ID NO: 42 - DNA, 53 bp, Artificial Sequence
  Description of Artificial Sequence Synthetic primer
cgtagtctggcacgtcgtatgggtacattttgccggtacgataatcaaagctg

SEQ ID NO: 43 - DNA, 23 bp, Artificial Sequence
  Description of Artificial Sequence Synthetic primer
cgtagtctggcacgtcgtatggg

SEQ ID NO: 44 - DNA, 18 bp, Artificial Sequence
  Description of Artificial Sequence Synthetic primer
tacccatacgacgtgcca

SEQ ID NO: 45 - DNA, 22 bp, Artificial Sequence
  Description of Artificial Sequence Synthetic primer
tggtgctcgagtttattcatgc

SEQ ID NO: 46 - protein, 70 aa, Artificial Sequence
  Description of Artificial Sequence Synthetic polypeptide
MAEDWLDCPALGPGWKRREVFRKSGATCGRSDTYYQSPTGDRIRSKVELTRYLGPACDLTLFDFKQGILC

SEQ ID NO: 47 - protein, 70 aa, Artificial Sequence
  Description of Artificial Sequence Synthetic polypeptide
MAESWQDCPALGPGWKRRESFRKSGASFGRSDIYYQSPTGEKIRSKVELTRYLGPACDLTLFDFRQGTLC

SEQ ID NO: 48 - protein, 69 aa, Artificial Sequence
  Description of Artificial Sequence Synthetic polypeptide
ESGKRMDCPALPPGWKKEEVIRKSGLSAGKSDVYYFSPSGKKFRSKPQLARYLGNAVDLSSFDFRTGKM

SEQ ID NO: 49 - protein, 72 aa, Artificial Sequence
  Description of Artificial Sequence Synthetic polypeptide
GSDKQGRTDCPALPPGWKKEEVIRKSGLSAGKSDVYYFSPSGKKFRSKPQLARYLGNAVDLSCFDFRTGKMM

SEQ ID NO: 50 - protein, 73 aa, Artificial Sequence
  Description of Artificial Sequence Synthetic polypeptide
ATAGTECRKSVPCGWERVVKQRLFGKTAGRFDVYFISPQGLKFRSKSSLANYLHKNGETSLKPEDFDFTVLSK

SEQ ID NO: 51 - protein, 73 aa, Artificial Sequence
  Description of Artificial Sequence Synthetic polypeptide
STTATEGHKPVPCGWERVVKQRLSGKTAGKFDVYFISPQGLKFRSKRSLANYLLKNGETFLKPEDFNFTVLPK

SEQ ID NO: 52 - protein, 91 aa, Artificial Sequence
  Description of Artificial Sequence Synthetic polypeptide
ASASPKQRRSIIRDRGPMYDDPTLPEGWTRKLKQRKSGRSAGKYDVYLINPQGKAFRSKVELIAYFEKVGDTSLDPNDFDFTVTGRGSPSR